(19) United States
(12) Patent Application Publication
(10) Pub. No.: US 2021/0393745 A1
(43) Pub. Date: Dec. 23, 2021

(54) OLIGOMER EXTENDED INSULIN-FC CONJUGATES AND THEIR MEDICAL USE

(71) Applicant: Novo Nordisk A/S, Bagsvaerd (DK)

(72) Inventors: Tina Moeller Tagmose, Ballerup (DK); Peter Madsen, Bagsvaerd (DK); Thomas Boerglum Kjeldsen, Virum (DK); Lone Pridal, Greve (DK); Zhaosheng Lin, Markham (CA); Yuanyuan Zhang, Beijing (CN); Zhe Wan, Beijing (CN)

(21) Appl. No.: 17/284,332

(22) PCT Filed: Oct. 9, 2019
(86) PCT No.: PCT/EP2019/077276

§ 371 Date: Apr. 9, 2021

(30) Foreign Application Priority Data
Oct. 10, 2018 (CN) PCT/CN2018/109562
Nov. 6, 2018 (EP) 18204512.0

(51) Int. Cl. A61K 38/28; A61K 47/22; C07D 207/46; A61K 47/68; A61P 3/10

(52) U.S. Cl. CPC A61K 38/28; A61K 47/22; A61P 3/10; A61K 47/6811; C07D 207/46

(57) ABSTRACT

This invention is in the field of protein conjugates. More specifically the invention relates to oligomer extended insulins with covalently attached Fc monomer polypeptides, for use in the treatment of a metabolic disorder or condition, and to methods of producing such oligomer extended insulin-Fc conjugates. The invention also relates to a novel Fc fragment, to intermediate products, and to the use of such intermediate products in processes for the synthesis of the oligomer extended insulin-Fc conjugates of the invention. Finally the invention provides pharmaceutical compositions comprising the oligomer extended insulin-Fc conjugates of the invention, and relates to the use of such compositions for the treatment or prevention of medical conditions relating to metabolic disorders or conditions.

### CROSS-REFERENCE TO RELATED APPLICATIONS

This application is a 35 U.S.C. § 371 National Stage application of International Application PCT/EP2019/077276 (WO2020/074544), filed Oct. 9, 2019, which claims priority to Chinese Patent Application PCT/CN2018/109562, filed Oct. 10, 2018 and European Patent Application 18204512.0, filed Nov. 6, 2018; the contents of which are incorporated herein by reference

### TECHNICAL FIELD

This invention is in the field of protein conjugates. More specifically the invention relates to oligomer extended insulins with covalently attached Fc monomer polypeptides, for use in the treatment of a metabolic disorder or condition, and to methods of producing such oligomer extended insulin-Fc conjugates.

The invention also relates to intermediate products, and to the use of such intermediate products in processes for the synthesis of the oligomer extended insulin-Fc conjugates of the invention.

Finally the invention provides pharmaceutical compositions comprising the oligomer extended insulin-Fc conjugates of the invention, and relates to the use of such compositions for the treatment or prevention of medical conditions relating to metabolic disorders or conditions.

### BACKGROUND

Oligomer extended insulins are insulins created by extending the A-chain and/or the B-chain of insulins with oligomers (made up of amino acid residues).

Fc-fusion proteins (sometimes called peptibodies) are chimeric proteins typically generated by fusing (i.e. joining two or more genes that originally coded for separate proteins) a biologically active polypeptide with the fragment crystallisable region (Fc-domain) of immunoglobulin G, and fusion proteins often combine the properties of their component parts, e.g. the IgG-like property of long serum half-life by virtue of binding to the neonatal Fc receptor, FcRn.

Protein conjugates, on the other hand, are compounds having a “large”, typically recombinant, effector molecule (such as e.g. IgG-Fc or albumin), covalently bound to a therapeutic polypeptide via a synthetic linker (e.g. a PEG linker). Protein conjugates are useful in multiple situations, and the identification and development of biological therapeutic compounds of increasing complexity have increased the focus on attractive methods for preparing such compounds. Difficulties with linkage of two or more proteins arise as proteins are not as stable as traditional chemical moieties, and traditional reaction chemistry may not be applied without damaging the proteins. Furthermore, linking two or more proteins is complicated by selectivity issues.

The half-lives of proteins and peptides may be extended by fusion to Fc. However, as insulin is a two chain polypeptide, that is expressed as a single chain analogue followed by enzymatic processing to obtain the two chains polypeptide, it is not ideal for protein fusions. Furthermore fusions are limited to the N- and/or C-terminal. Therefore, e.g. in order to extend the half-life of insulin by the action of Fc, a chemical conjugation is needed.

When conjugating two proteins, the properties of the linker are important. A short linker might impact the activities of the respective proteins. Bringing the two proteins too close might impact the biophysical stability of the molecule. Linker properties which can affect this could be e.g. the position of conjugation on second molecule, length of linker, flexibility of linker, polarity of linker. In some cases a flexible linker is preferred and in other cases a stiff linker is needed. Conjugating molecules/proteins to a second molecule might have a negative impact on the biophysical properties of the conjugate, which might be solved by the linker properties.

WO 2011/122921 describes an insulin conjugate having improved in vivo duration, which conjugate is prepared by covalently linking insulin with an immunoglobulin Fc region via a hydrophilic non-peptidyl (e.g. PEG) linker, having a reactive group (e.g. aldehyde functionalities as propionaldehyde) at both ends, a so called homo-bifunctional linker. The PEG linkers used are polydisperse and of 3.4 KDa to 10 KDa in size, and the use of polydisperse linkers causes challenges for the synthesis and analysis of the final product.

WO 2016/178905 describes fusion proteins comprising an insulin receptor agonist fused to a human IgG Fc region through the use of a peptide linker.

WO 2016/193380 describes novel insulins or insulins analogues that are extended with sequences of predominantly polar amino acid residues in order to improve the half-life and stability of the drug substance.

The most common way to obtain site specific conjugates between proteins and peptides, small molecules or protractor polymers, is to exploit the unique nucleophilic properties of thiols of Cys and primary amino groups of the N-terminal amino acid, and the epsilon-NH₂ of Lys side chains, as reactive handles. Lysine is usually abundant in proteins (Fc contains more than 30 lysine residues), restricting the use of lysine as a chemical handle. However, in the case of insulin, only one lysine is present (i.e. at position B29). Cysteine's, on the other hand, are less frequently found in proteins, and moreover usually engaged in forming disulphide bridges. Cysteine can be introduced by genetically engineering. However, in the case of insulin, this modification has proven to be difficult, in particular due to low expression yields and various folding issues.

Also challenging is to site-specifically conjugate two proteins together through their respective Cys, N-terminal amino group or Lys residues. The use of a hetero-bifunctional linker can serve this purpose. Even more challenging becomes, in turn, the task of conjugating two proteins, in which one protein is to be connected through both sulphur atoms derived from a reduced disulphide bond.

### SUMMARY

The oligomer extended insulin-Fc conjugates of the invention display reduction of insulin receptor affinity, compared to similar non-conjugated and non-extended analogues. This reduction in insulin receptor affinity contributes to the protraction of the insulin-Fc conjugate in circulation, since insulin is internalised and degraded upon receptor activation. Hence, clearance of the insulin-Fc conjugate of the invention is reduced. This reduction of insulin receptor affinity probably does not cause a loss of potency, e.g., as measured in a standard hyperinsulinaemic euglycaemic clamp assay, and the combination of a high FcRn binding and a low insulin receptor affinity is considered beneficial for obtaining long duration of action of the insulin-Fc conjugates of the invention.

Accordingly, in its first aspect, the present invention provides novel oligomer extended insulin-Fc conjugates represented by Formula I:

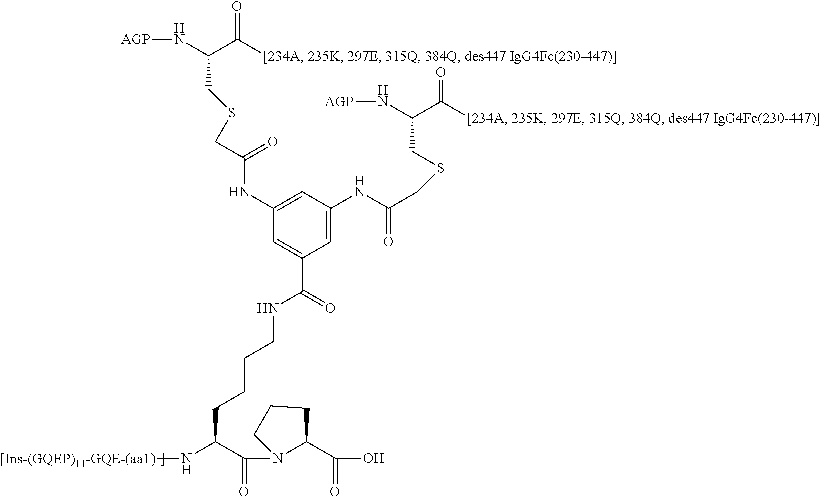

wherein Ins represents an analogue of human insulin, which insulin analogue is a two-chain insulin molecule comprising an A- and a B-chain, and which insulin analogue comprises the amino acid substitutions A14E, A21G, B25H, B29R, and the deletion desB30;

wherein (GQEP)11-GQE-(aa1) is a recombinant extension fused to the C-terminus of the insulin A-chain; and

wherein (aa1) is absent or proline (P).

In a second aspect, the invention provides an intermediate compound Ins-(GQEP)₁₁-GQE-(aa1)-KP,

wherein Ins represents an analogue of human insulin, which insulin analogue is a two-chain insulin molecule comprising an A- and a B-chain, and which insulin analogue comprises the amino acid substitutions A14E, A21G, B25H, B29R, and the deletion desB30;

wherein (GQEP)11-GQE-(aa1) is a recombinant extension fused to the C-terminus of the insulin A-chain; and

wherein (aa1) is absent or proline (P).

In a third aspect, the invention provides an intermediate compound of Formula II:

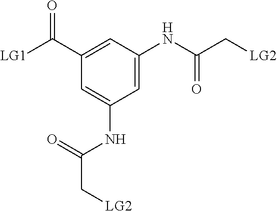

wherein

LG1 represents a leaving group reactive towards primary amino groups; and

LG2 represents the leaving group of a thiol reactive group.

In a fourth aspect, the invention provides an intermediate compound of Formula III:

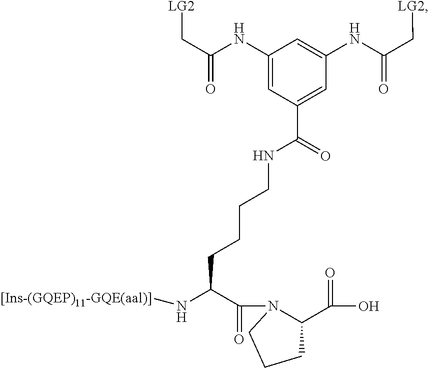

wherein Ins represents an analogue of human insulin, which insulin analogue is a two-chain insulin molecule comprising an A- and a B-chain, and which insulin analogue comprises the amino acid substitutions A14E, A21G, B25H, B29R, and the deletion desB30;

wherein (GQEP)11-GQE-(aa1) is a recombinant extension fused to the C-terminus of the insulin A-chain;

wherein (aa1) is absent or proline (P); and

wherein each LG2 represents the leaving group of a thiol reactive group.

In a fifth aspect, the invention provides an intermediate compound which is 226A, 227G, 228P, 234A, 235K, 297E, 315Q, 384Q, des447 hIgG4-Fc(226-447).

In a sixth aspect, the invention provides a pharmaceutical composition comprising the insulin-Fc conjugate of the invention and one or more pharmaceutically acceptable carriers or diluents.

In a seventh aspect, the invention provides an insulin-Fc conjugate of the invention for use as a medicament.

In an eighth aspect, the invention provides an insulin-Fc conjugate of the invention for use in the treatment or alleviation of a disease or disorder or condition of a living animal body, including a human, which disease, disorder or condition may be selected from a disease, disorder or condition relating to diabetes, Type 1 diabetes, Type 2 diabetes, impaired glucose tolerance, hyperglycemia, dyslipidemia, obesity, metabolic syndrome (metabolic syndrome X, insulin resistance syndrome), hypertension, cognitive disorders, atherosclerosis, myocardial infarction, stroke, cardiovascular disorders, coronary heart disease, inflammatory bowel syndrome, dyspepsia, or gastric ulcers.

The insulin-Fc conjugates of the invention show improved pharmacokinetic and/or pharmacodynamic profiles.

In one aspect of the invention the compounds of the invention show increased pharmacodynamic potencies.

In another aspect of the invention the compounds of the invention show at least two fold increased pharmacodynamic potencies compared with currently marketed long-acting insulins.

In another aspect of the invention the compounds of the invention have increased in vivo half-life.

### BRIEF DESCRIPTION OF DRAWINGS

FIG. 1 shows the blood glucose lowering effect of Examples 5.1, 5.2 and 5.3. The compounds were administered s.c. to fed Sprague-Dawley rats in a dose of 30 nmol/kg.

FIG. 2 shows the baseline corrected blood glucose lowering effect of Examples 5.1, 5.2 and 5.3. The compounds were administered s.c. to fed Sprague-Dawley rats in a dose of 30 nmol/kg.

FIG. 3 shows the pharmacokinetic profiles of Examples 5.1, 5.2 and 5.3. The compounds were administered s.c. to fed Sprague-Dawley rats in a dose of 30 nmol/kg.

FIG. 4 shows the blood glucose lowering effect of Examples 5.1, 5.2 and 5.3. The compounds were administered s.c. to fed streptozotocin treated Sprague-Dawley rats in a dose of 30 nmol/kg.

FIG. 5 shows the baseline corrected blood glucose lowering effect of Examples 5.1, 5.2 and 5.3. The compounds were administered s.c. to fed streptozotocin treated Sprague-Dawley rats in a dose of 30 nmol/kg.

FIG. 6 shows the pharmacokinetic profiles of Examples 5.1, 5.2 and 5.3. The compounds were administered s.c. to fed streptozotocin treated Sprague-Dawley rats in a dose of 30 nmol/kg.

### DESCRIPTION

We provide herein novel oligomer extended insulin-Fc conjugates represented by Formula I:

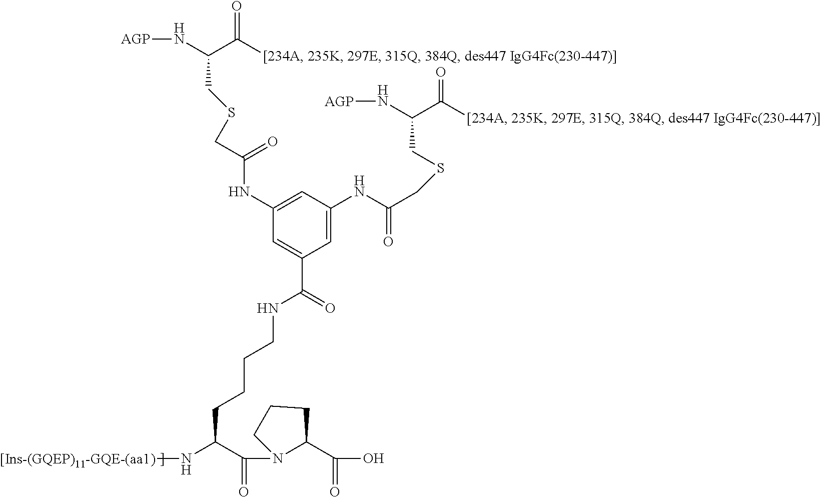

wherein Ins represents an analogue of human insulin, which insulin analogue is a two-chain insulin molecule comprising an A- and a B-chain, and which insulin analogue comprises the amino acid substitutions A14E, A21G, B25H, B29R, and the deletion desB30;

wherein (GQEP)11-GQE-(aa1) is a recombinant extension fused to the C-terminus of the insulin A-chain; and wherein (aa1) is absent or proline (P).

### Insulin Analogues

Human insulin consists of two polypeptide chains, i.e. the A-chain (a 21 amino acid peptide, SEQ ID NO:1) and the B-chain (a 30 amino acid peptide, SEQ ID NO:2), respectively, interconnected by two cysteine disulphide bridges. A third intra chain disulphide bridge is present in the A-chain.

Herein, the term insulin covers natural occurring insulins, e.g. human insulin, as well as analogues hereof. The numbering of the amino acid positions in insulin analogues, insulins and A- and B-chains, is done relative to human insulin.

Herein, the term insulin analogue covers a modified human insulin polypeptide which has a molecular structure which formally can be derived from the structure of a naturally occurring insulin, e.g. human insulin, by deleting and/or substituting (replacing) one or more amino acid residue occurring in the natural insulin, and/or by adding one or more amino acid residues.

In one embodiment an insulin analogue comprises less than 10 amino acid modifications (substitutions, deletions, additions (including insertions) and any combination thereof) relative to human insulin, alternatively less than 9, 8, 7, or 6 modifications relative to human insulin. In addition to these modifications, the Fc-Insulin conjugates of the present invention comprises a recombinant extension fused to the insulin A-chain C-terminus. Herein, the recombinant extension is not defined as part of the insulin analogue.

Herein terms like A1, A2, A3 etc. indicate position 1, 2 and 3, respectively, in the A-chain of insulin, when counted from the N-terminal end. Similarly, terms like B1, B2, B3 etc. indicates position 1, 2 and 3, respectively, in the B-chain of insulin, when counted from the N-terminal end. Using the established one letter codes for amino acids, terms like A21A, A21G and A21Q designate that the amino acid in the A21 position is A, G and Q, respectively. Using the established three letter codes for amino acids, the corresponding expressions are AlaA21, GlyA21 and GlnA21, respectively.

Herein terms like desB30 indicate an insulin analogue lacking the B30 amino acid residue.

The term A22 indicate the position of the amino acid C-terminally to A21. In this way the term A23 indicates the position of the first amino acid C-terminally to A22. Thus A24 indicate the position of the amino acid C-terminally to A23, and so forth. In this way A22G, A23G indicates that the C-terminal of the A chain has been extended with a glycine (G) residue C-terminally to position A21, followed by a glycine (G) residue located C-terminally to the glycine (G) residue at position A22 (A22G).

The insulin analogue according to the present invention is an analogue of human insulin, which insulin analogue comprises the amino acid substitutions A14E, A21G, B25H, B29R, and the deletion desB30.

In one embodiment, the insulin analogue of the present invention is an analogue of human insulin, which insulin analogue comprises the amino acid substitutions A14E, A21G, B25H, B29R, and the deletion desB30, and also further comprises the amino acid substitution B16H or the amino acid substitution B16E.

In further embodiments, the insulin analogue for use according to the invention is selected from the following examples:

[A14E, A21G, B25H, B29R, desB30] human insulin (A-chain of SEQ ID NO: 3, B-chain of SEQ ID NO:4);

[A14E, A21G, B16H, B25H, B29R, desB30] human insulin (A-chain of SEQ ID NO: 3, B-chain of SEQ ID NO:5); and

[A14E, A21G, B16E, B25H, B29R, desB30] human insulin (A-chain of SEQ ID NO: 3, B-chain of SEQ ID NO:6).

### Oligomer Extended Insulin Analogues

The insulin-Fc conjugates of the present invention comprises a recombinant extension, which extension is made up of repeats of the amino acid sequence GQEP followed by an amino acid sequence selected from the group of GQEKP and GQEPKP.

The recombinant extension according to the present invention is an extension fused to the insulin A-chain C-terminus, which extension has the amino acid sequence (GQEP)₁₁-GQE-(aa1)-KP, wherein (aa1) is absent or proline (P).

In one embodiment, the extension is selected from (GQEP)₁₂-KP and (GQEP)₁₁-GQEKP.

In one embodiment, the extension is (GQEP)₁₂-KP.

In another embodiment, the extension is (GQEP)₁₁-GQEKP.

It shall be noted that the extension introduced according to the invention is not intended for, and does not contribute significantly to the extended half-life observed for the insulin-Fc conjugates of the invention, but in the context of the present invention, the extension happens to provide a suitable spacing group/linker, thereby avoiding shielding of the insulin analogue, and the extension also contributes to a better (improved) solubility and/or improved glycodynamic potency.

### Nomenclature of Oligomer Extended Fusion Insulin Analogues

In the context of this invention, the oligomer extended insulin analogues are named relative to human insulin by specification of amino acid deletions, substitutions, insertions and extensions.

In this way the compound of Example 1.1 represents an analogue of human insulin (A14E, A21G, B25H, B29R, desB30), wherein the naturally occurring amino acid residues located in position A14 and A21 of the A-chain has been substituted for glutamic acid (E) and glycine (G) respectively, and wherein the naturally occurring amino acid residues located in position B25 and B29 have been substituted for histidine (H) and arginine (R), respectively, and position B30 has been deleted, and which analogue has been extended C-terminally from the A-chain, starting at amino acid position A22, with an extension made up of twelve repeats of the four amino acid residues (GQEP), and in the specified order, to make up an extension consisting of a total of 48 amino acids residues (designated (GQEP)₁₂), followed by KP. The full extension may be designated as (GQEP)₁₂—KP (and the lysine is thus referred to as A70K).

This analogue may also be designated A14E, A21G, A22(GQEP)₁₂, A70K, A71P, B25H, B29R, desB30 human insulin (A-chain of SEQ ID NO:10 and B-chain of SEQ ID NO:4), and the compound is illustrated in Chem. 1 below. A22(GQEP)₁₂ thus designates a (GQEP)₁₂ extension attached to A21, wherein the first amino acid of the extension (i.e. the amino acid attached to A21, in this example G) corresponds to the A22 position, i.e. in this example A22G.

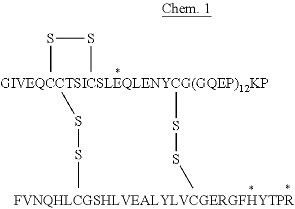

### The Linker

In the context of this invention a linker is a chemical moiety or residue used to covalently link the proteins in question. As the linker reacts with the proteins, a linker radical is formed. The term “-linker-” is thus intended to mean the chemical unit of the insulin-Fc conjugate, which is covalently linked to an amino acid residue of each of the polypeptides of the protein conjugate.

According to this invention, the linker is used for linkage of an insulin analogue to an Fc-domain. The Fc-domain consists of two polypeptides that are usually held together by covalent and/or non-covalent bonds including inter-polypeptide disulphide-bond(s). Covalent linkage using a conventional bi-valent linker would link the protein to only one of the Fc polypeptides. However, using the trivalent linker (also referred to as tri antennary linker) according to the present invention, a protein conjugate where both of the two Fc polypeptide chains are linked to the insulin analogue is obtained.

The linker of the present invention is illustrated by Chem. 2 below.

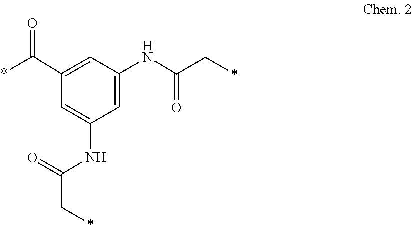 [SMILES: *CC(=O)Nc1cc(NC(=O)C*)cc(C(*)=O)c1]

wherein # denotes the attachment point to the epsilon amino group of the lysine (K) residue in the recombinant extension of insulin; and * denotes the attachment point to the sulphur atoms of the cysteine (C) residues in position 229 of the Fc component, i.e. 226A, 227G, 228P, 234A, 235K, 297E, 315Q, 384Q, des447 hIgG4-Fc(226-447).

### The Antibody Fc Component

The Fc region is a C-terminal region of an IgG heavy chain, which is responsible for binding to Fc receptors that undertakes various functions, including recycling, which results in prolonged half-life. The Fc portion is typically derived from IgG, and conjugate moieties often include portions of the immunoglobulin sequence that include the neonatal Fc receptor (FcRn) binding site. FcRn, a salvage receptor, is responsible for recycling of immunoglobulins and returning them to circulation in blood. Mutations of the immunoglobulin Fc region are often introduced in order to modify certain properties, e.g. to obtain increased affinity for FcRn, to obtain prolonged half-life, or to reduce or increase binding to other receptors, to obtain reduced or increased immune effector functions.

The antibody isotype IgG is further grouped into subclasses (e.g. human IgG1, IgG2, IgG3 and IgG4) based on additional small differences in their amino acid heavy chain sequences.

In the context of this invention, the term “Fc region”, “Fc fragment” or “Fc-domain” refers to the fragment crystallisable of an antibody. The Fc region is the tail of an antibody. For IgG antibodies, the Fc region contains two identical polypeptides (i.e. is a homo dimer), both comprising the second and third constant domains (CH2 and CH3) of the heavy chain. The Fc-domain may also be referred to as a dimer, as the two Fc polypeptides interact non-covalent and possibly also covalently, as hinge cysteine's may form disulphide bond(s). In the context of this invention, one Fc (monomeric) polypeptide of the Fc-domain is referred to as a “Fc monomer” or “Fc polypeptide”. Also in the context of this invention, the protein sequences of the Fc-domain are referred to as “Fc polypeptides”, and comprise at least the CH2 and CH3 domains.

The hinge region is the protein segment between CH1 and CH2 of the constant region of the antibody. The hinge region corresponds to position 216 to 238 in the case of IgG1 and 219 to 238 in case of IgG4-Fc according to Kabat EU numbering. The hinge region can further be divided into the upper hinge region corresponding to position 216 to 225 and 219 to 225 in the case of IgG1 and IgG4, respectively. The core hinge region is referred to as position 226 to 231 in both the case of IgG1 and IgG4, and the lower hinge region is referred to as position 232 to 238.

The Fc region of human antibodies is glycosylated, and glycosylation is believed to be involved in C1q interactions, therefore C1q binding may be decreased by removing the glycosylation. Furthermore, aglycosylated Fc have diminished or weak binding to the Fc gamma receptors I, IIa, Ilb, and IIIa, respectively, which again allows for low Antibody-Dependent Cell-mediated Cytotoxicity (ADCC) and/or complement-dependent cytotoxicity (CDC).

Glycosylation may be removed enzymatically. Production of Fc in E. coli results in aglycosylated Fc. Techniques for the preparation of such sequence derivatives of the immunoglobulin Fc region are well known in the art, and are disclosed in e.g. WO 97/34631 and WO 96/32478.

The IgG derived Fc-fragment of the present invention is an aglycosylated hIgG4 Fc-fragment, which naturally have weak binding to the Fc gamma receptor III.

The Fc component according to the present invention is 226A, 227G, 228P, 234A, 235K, 297E, 315Q, 384Q, des447 hIgG4-Fc(226-447) (SEQ ID NO:7).

The Fc component is modified so that the hinge region of the Fc polypeptide comprises only one (native) cysteine (C) residue. This was achieved by starting the Fc polypeptide sequence of this invention at core hinge position 226 and by substituting the native cysteine (C) residue at position 226. This cysteine of the first Fc monomer is capable of forming a disulphide bond with the similar cysteine residue, located on the second monomer of the original (homo dimer) Fc polypeptide, e.g. as illustrated in Chem. 3, below. The Fc polypeptides of the Fc-domain may thus be covalently linked by a di-sulphide bridge or, alternatively, be non-covalently linked.

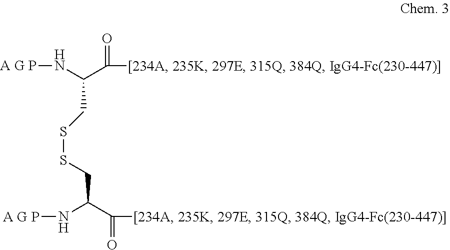

The linkages to the Fc polypeptides of the invention take place via sulphur atoms (—S—) derived from a cysteine residue in the Fc polypeptides, as described above.

The hinge core sequence (positions 226-231) of native human IgG4 is CPSCPA (SEQ ID NO:8).

The hinge core sequence of the present invention has been modified to make the Fc region expressed as homogenous product via efficient removal of initial Met without extra cleavage of following residues. The hinge core sequence according to the invention is AGPCPA (SEQ ID NO:9). The IgG4 derived Fc-domain for use according to the invention thus comprises the amino acid modifications 226A, 227G and 228P.

The constant region is modified to stabilize the molecule and to alter one or more of its functional properties, such as e.g. serum half-life, complement fixation, Fc-receptor binding, protein stability and/or antigen-dependent cellular cytotoxicity, or lack thereof. Furthermore, the Fc-domain may be chemically modified (e.g. one or more chemical moieties can be attached to the Fc part) to alter its glycosylation, to also alter one or more functional properties of the antibody.

The IgG4 derived Fc-domain for use according to the invention comprises the amino acid substitutions F234A and L235K, which results in decreased affinity to certain Fc gamma receptors.

In order to improve chemical stability of the Fc-domain, asparagines, prone to deamidation, may be substituted by glutamine, aspartic acid or glutamic acid. The Fc component of the invention comprises the N297E, N315Q and N384Q substitution.

In order to improve physical stability of the Fc-domain, The Fc component of the invention comprises the L235K substitution.

Furthermore, the C-terminal lysine of IgG's is cleaved in vivo in the human blood, and endogenous IgG isolated from human blood contains very low levels of C-terminal lysine. Therefore the C-terminal lysine at position 447 is deleted, e.g. the Fc component of the invention includes des447.

EU Index of Kabat

The (Kabat) EU numbering scheme is a widely adopted standard for numbering the residues in an antibody in a consistent manner, developed on the basis of sequence alignment. In the context of this application the numbering of the residues in the Fc chain is done according to the EU index of Kabat (as described in Kabat et al., Sequences of Proteins of Immunological Interest, 5th Ed. Public Health Service, National Institutes of Health, Bethesda, Md. (1991)).

According to this invention, the native sequence of human IgG4-Fc starting at position 226 and ending at position 447 is designated hIgG4-Fc(226-447). As Fc consists of two identical polypeptides, only the sequence of one polypeptide is given. If serine (S) at position 228 is mutated into proline (P), the analogue is designated 228P hIgG4-Fc(226-447). Likewise, if cysteine (C) at position 226 is mutated into alanine (A), proline (P) at position 227 is mutated into glycine (G), and serine (S) at position 228 is mutated into proline (P), this product is designated 226A, 227G, 228P hIgG4-Fc(226-447).

As described above, the insulin-Fc conjugates of the invention may be described as two monomer Fc polypeptides covalently joined with insulin via the linker described above.

### Insulin-Fc Conjugates of the Invention

In the context of this invention, insulin-Fc conjugates represent compounds including an insulin analogue and two Fc monomers, which are covalently linked via a linker.

The present invention provides novel insulin-Fc conjugates represented by Formula I:

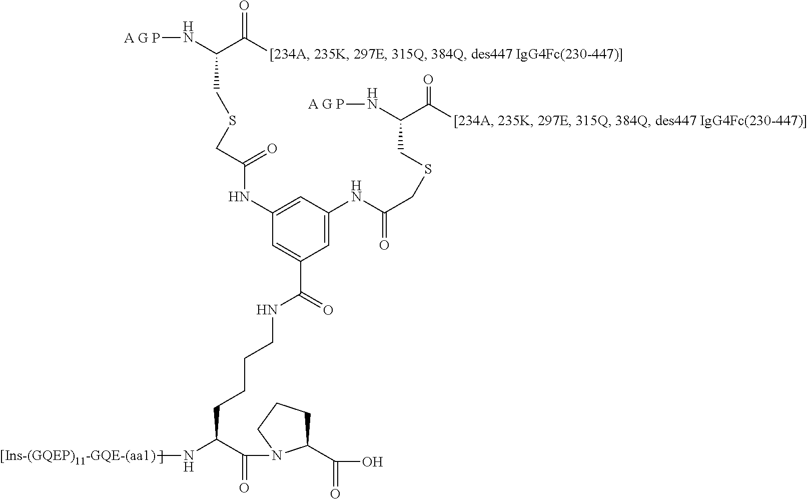

wherein Ins represents an analogue of human insulin, which insulin analogue is a two-chain insulin molecule comprising an A- and a B-chain, and which insulin analogue comprises the amino acid substitutions A14E, A21G, B25H, B29R, and the deletion desB30;

wherein (GQEP)11-GQE-(aa1) is a recombinant extension fused to the C-terminus of the insulin A-chain; and

wherein (aa1) is absent or proline (P).

In further embodiments, the insulin-Fc conjugate according to the invention is selected from the following examples:

(A14E, A21G, A22(GQEP)₁₂, A70K\hat{ }, A71P, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl conjugate (compound of Example 5.1);

(A14E, A21G, A22(GQEP)₁₁, A66G, A67Q, A68E, A69K\hat{ }, A70P, B16H, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl conjugate (compound of Example 5.2); and A14E, A21G, A22(GQEP)₁₁, A66G, A67Q, A68E, A69K\hat{ }, A70P, B16E, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl conjugate (compound of Example 5.3).

### Nomenclature of the Insulin-Fc Conjugates of the Invention

In the context of this invention, and for ease of information, the insulin-Fc conjugates of the invention are designated according to the peptide component parts (i.e. the insulin component and the immunoglobulin Fc component) constituting the conjugate of the invention, along with a specification of the linking group.

In this way the insulin-Fc conjugate of Example 5.1 (presented below as Chem. 4), which represents a conjugate linked through an A-chain C-terminal extension, may be designated as an (A14E, A21G, A22(GQEP)₁₂, A70K\hat{ }, A71P, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl conjugate, indicating that the compound is made up of an insulin component (i.e. A14E, A21G, A22(GQEP)₁₂, A70K, A71P, B25H, B29R, desB30 human insulin) and an Fc component (i.e. 226A, 227G, 228P, 234A, 235K, 297E, 315Q, 384Q, des447 hIgG4-Fc(226-447)), conjugated via the trivalent linking group 3,5-bis[(2-acetyl)amino]benzoyl, and that the linker bridges the epsilon amino group of lysine A70K of the insulin component to —C(O)—, and each of the thiols of Cys229 of the Fc component polypeptides to —C(O)—CH₂—.

Note that A70K\hat{ } indicates attachment point of the linker to insulin. A thus indicates the position in the extended insulin analogue to which the linker is attached.

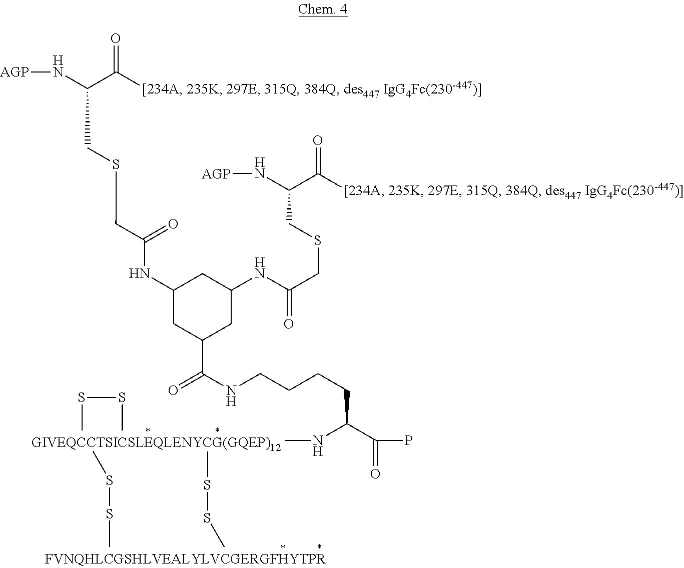

In Chem. 4 the first three amino acids (i.e. A-G-P) of both Fc polypeptide sequences are shown, and the fourth amino acids (i.e. Cys) are shown expanded. The substitutions and the deletion in the remaining part of the Fc polypeptides (i.e. positions 230 to 447) are indicated in the brackets [e.g. 234A, 235K, 297E, 315Q, 384Q, des447].

### Intermediate Products

In further aspects, the invention provides intermediate compounds for use in the manufacture of the insulin-Fc conjugate of the invention.

Oligomer Extended Insulin Analogues

The oligomer extended insulin analogues may be obtained by methods known in the art, e.g. as described in WO 2016/193380.

According to the present invention, the entire sequence of the oligomer extended insulin construct contains one lysine (K) residue only.

In one embodiment, the intermediate compound for use according to the invention is an insulin analogue comprising a polar recombinant extension fused to the insulin A-chain C-terminus, which extension has the amino acid sequence (GQEP)₁₁-GQE-(aa1)-KP, wherein aa1 is absent or proline (P); and the insulin is an analogue of human insulin, which insulin analogue comprises the amino acid substitutions A14E, A21G, B25H, B29R, and the deletion desB30.

In further embodiments the insulin analogue further comprises the amino acid substitution B16H or B16E.

In further embodiments, the intermediate compound for use according to the invention is selected from the following examples:

A14E, A21G, A22(GQEP)₁₂, A70K, A71P, B25H, B29R, desB30 human insulin (compound of Example 1.1);

A14E, A21G, A22(GQEP)₁₁, A66G, A67Q, A68E, A69K, A70P, B16H, B25H, B29R, desB30 human insulin (compound of Example 1.2); and

A14E, A21G, A22(GQEP)₁₁, A66G, A67Q, A68E, A69K, A70P, B16E, B25H, B29R, desB30 human insulin (compound of Example 1.3).

Fc Fragments

The insulin-Fc conjugates of the present invention involve the use of two monomer Fc polypeptides covalently joined with insulin via a linker.

In a further aspect, the invention provides an Fc polypeptide for use as an intermediate compound in the manufacture of an insulin-Fc conjugate of the invention.

The Fc polypeptide for use according to the invention is 226A, 227G, 228P, 234A, 235K, 297E, 315Q, 384Q, des447 hIgG4-Fc(226-447).

Linking Groups

In another aspect the present invention provides an intermediate compound for use in the manufacture of the insulin-Fc conjugate of the invention.

A tri-antennary linker is provided, in which the first terminus is capable of forming a stable covalent bond with the epsilon amino group of a lysine residue of the insulin component of Formula I, and which terminus remains unaffected by thiols, or by any other residue within the protein.

The second and third termini of the linker are identical, and capable of forming stable covalent bonds with thiol moieties (i.e. —SH) of the Fc component of Formula I (called the “Cys-reactive termin-us/-i”). After the first conjugation event has taken place (i.e. conjugation of the first terminus to lysine), the Cys-reactive termin-us/i can react directly (using e.g. bromo or iodo acetamides, Michael acceptors, etc., or can be amenable of a chemical transformation, such that it will make them reactive towards thiol(s) of a second protein (changing e.g. chloroacetamide to iodoacetamide), thereby giving the desired insulin-Fc conjugate of Formula I in a sequential, two-step fashion.

The particular design of the linker of the invention, containing two reactive ends that each comprises an Sp2 hybridised carbon C atom, a CH₂ group and a halogen (Hlg) atom (represented as (C═O)—CH₂—Hlg), is particularly well suited for conjugating inter-chain disulphides of Fc fragments, as these can be easily and selectively reduced to free thiols without interfering with other intra-chain disulphides.

The intermediate product of the invention represents a tri-antennary linker which allows for an efficient protein-protein conjugation by means of hetero-functional linkers. By use of different leaving groups, the linkage of reactants can be controlled, and the intermediate is particularly useful in a method of synthesis in order to covalently link two or more proteins in an ordered fashion, ensuring that different proteins can be attached at each end of the linker.

The intermediate compound for use according to the invention may be characterised by the general Formula II:

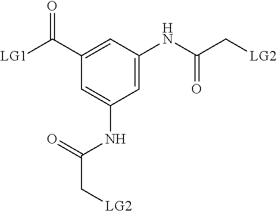

wherein

LG1 (that may also be designated as the Lys reactive terminus) represents a leaving group reactive towards primary amino groups; and

LG2 (that may also be designated as the Cys reactive terminus) represents the leaving group of a thiol reactive group.

In the context of this invention, LG1 represents a leaving group reactive towards primary amino groups such as of commonly used active esters. Such leaving groups include active esters conventionally used in peptide synthesis, including but not limited to, N-hydroxysuccinimide (NHS) esters, sulfo-NHS ester, pentafluorophenol (PFP) ester, p-nitrophenol (PNP) ester, hydroxybenzotriazole (HOBt) ester and ethyl (hydroxyimino)cyanoacetate ester (Oxyma).

In one embodiment, the “Lys reactive terminus” (LG1) is represented by a succinimide ester (OSu ester) activated carboxylic acid moiety, and the “Cys reactive termini” LG2 are each represented by iodide-, bromide- or chloride. After reaction of the epsilon amino group of the Lys positioned in the terminal part of the extension of insulin (i.e. the first protein) with the OSu ester, and appropriate purification, the resulting conjugate is reacted with the thiols of the Fc-fragments (i.e. the second and third protein) formed upon reduction of the inter chain disulphide, directly, or in the case of chloroacetamide, after exposure to high concentration of iodide ions, to bring about the so-called “Finkelstein reaction”, which reaction results in a chloro to iodo exchange, thus generating a bis-iodoacetamide moiety. The final result is the formation of a covalent, site-specific conjugate between the two proteins of interest, i.e. an insulin-Fc conjugate of Formula I.

In a further embodiment, the intermediate product is (2,5-dioxopyrrolidin-1-yl)-3,5-bis[(2-bromoacetyl)amino]benzoate shown in Chem. 5:

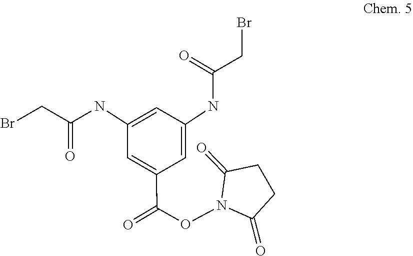

Oligomer Extended Insulin Analogues Comprising Linking Group

In one embodiment, an intermediate compound according to the invention is a compound characterised by the general Formula III:

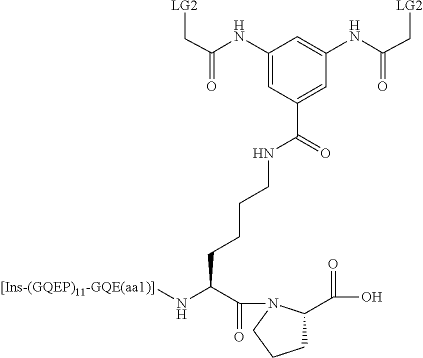

wherein,

wherein Ins represents an analogue of human insulin, which insulin analogue is a two-chain insulin molecule comprising an A- and a B-chain, and which insulin analogue comprises the amino acid substitutions A14E, A21G, B25H, B29R, and the deletion desB30; wherein (GQEP)11-GQE-(aa1) is a recombinant extension fused to the C-terminus of the insulin A-chain;

wherein (aa1) is absent or proline (P); and

wherein each LG2 represents the leaving group of a thiol reactive group.

In a further embodiment, each LG2 represents Br.

### Methods for the Preparation of Intermediate Products

The linker for use according to the invention may be produced by standard technologies, e.g. as described in the working examples below.

The proteins to be conjugated and the linker may be prepared and purified separately.

In one aspect, one end of the linker is a primary amino group reactive group, and the other end has two thiol reactive groups.

In a third embodiment, a primary amino group of insulin is reacted with the linker, followed by reaction with an Fc with two free cysteines.

In a further embodiment, Fc is connected through both sulphur atoms derived from a reduced disulphide bond.

### Methods for the Preparation of Insulin-Fc Conjugates

In a further aspect, the invention provides a method for preparing the insulin-Fc conjugates of the invention.

The method of the invention comprises the (consecutive) steps of
A) Preparation of the intermediate compound of Formula II;
B) Coupling the oligomer extended insulin analogue construct to the intermediate of Formula II to obtain the intermediate compound of Formula III;
C) Reduction of the inter-chain disulphide bond of Fc to obtain two Fc monomers, each holding a free cysteine; and
D) Coupling the two Fc monomers to the intermediate of Formula III to obtain the insulin-Fc conjugate of Formula I.

Generally the individual components, i.e. the oligomer extended insulin analogue component, the Fc component, and the linker, are produced separately and coupled together under suitable reaction conditions.

The insulin components for incorporation into the insulin-Fc conjugate according to the invention may be obtained by conventional methods for the preparation of insulin, insulin analogues and insulin derivatives, e.g. as outlined in WO 2008/034881, or in WO 2016/193380.

Fc domains may be obtained from full length antibodies isolated from humans or animals, or may be produced recombinant and obtained from transformed mammalian cells or microorganisms. Multiple technologies to obtain Fc-domains are known in the art.

An Fc-domain can be produced from a full length antibody by digestion with a proteolytic enzyme such as papain or pepsin. Affinity chromatography and DEAE anion-exchange chromatography can be used to separate the resulting Fab and F(ab′)₂ from the Fc-domain. Based on SEC-HPLC analysis, the purity of the Fc-fragment can be determined.

When recombinant methods are used, the desired polypeptide can be expressed and the Fc domain subsequently purified. In one embodiment the Fc domain is a human-derived Fc-domain, such as a human IgG Fc-domain obtained from transformed microorganisms or mammalian cells.

In addition, the Fc-fragment of the present invention may be in the form of having native sugar chains, increased sugar chains compared to a native form or decreased sugar chains compared to the native form, or may be in an aglycosylated form. The increase, decrease or removal of sugar chains of the Fc-fragment may be achieved by methods known in the art, such as a chemical method or an enzymatic method. Otherwise the asparagine at position 297, which is the natural glycosylation site can be mutated by molecular engineering to e.g. alanine. In the case where the recombinant DNA technology method is using the microorganism E. coli, the Fc produced will be aglycosylated.

The method described herein is suitable for preparing protein conjugates when at least one of the proteins to be conjugated includes a free cysteine. A free cysteine is a cysteine residue available for conjugation via a thiol reactive linking. A free Cys is usually a cysteine residue that does not engage in intra protein di-sulphide bonds. Frequently the free cysteine need to be liberated prior to the conjugation reaction, as proteins with a free Cys may form mixed disulphide with other sulphur containing molecules, usually small organic molecules present in the cell extract when the protein is produced and purified.

Free cysteine's may also be generated by reducing an existing disulphide bond, which will make available two free cysteine's, using reducing agents including trialkylphosphines such as TCEP, BSPP or the like, or with thiols such as DTT, mercapto ethanol and the like.

In one embodiment, two equivalent cysteines may be generated by reduction of an Fc-domain comprising a disulphide bond adjoining the two monomer polypeptides of the Fc-domain.

In a further embodiment, the Fc-domain comprises a single inter chain disulphide bond in the hinge region of the Fc-domain, e.g. in the 229 position of an IgG1-Fc polypeptide or an IgG4-Fc polypeptide fragment. Such a fragment may be linked with the two arms of the trivalent linker using methods described herein. The resulting protein conjugation (or conjugate intermediate) will have a bi-functional symmetrical) linkage with the Fc-domain (Fc polypeptide), and a third arm conjugated with the insulin component [Ins-(GQEP)₁₁-GQE(aa1)KP].

As described herein, the linker is covalently bound to the insulin analogue via the epsilon amino group of a lysine. Lysines are usually abundant in proteins and therefore not suitable for selective conjugation. However, the insulin analogue for use according to the invention only holds one lysine residue.

In one embodiment, a lysine residue located at the C-terminus sequence Lys-Pro (KP) end of an A-chain extension is used as conjugation site.

### Pharmaceutical Compositions

The present invention relates to insulin-Fc conjugates useful as medicaments, and in particular for use in the treatment, prevention or alleviation of a metabolic disease or disorder or condition.

Pharmaceutical compositions comprising the insulin-Fc conjugate of the invention or a pharmaceutically acceptable salt, amide, or ester thereof, and a pharmaceutically acceptable excipient may be prepared according to methods known in the art.

Therefore, in another aspect, the invention provides novel pharmaceutical compositions comprising a therapeutically effective amount of an insulin-Fc conjugate of the present invention, optionally together with one or more adjuvants, excipients, carriers and/or diluents.

The term “excipient” broadly refers to any component other than the active therapeutic ingredient(s). The excipient may be an inert substance, an inactive substance, and/or a not medicinally active substance.

Injectable compositions may be prepared by using conventional techniques, which typically includes dissolving and mixing the ingredients as appropriate to give the desired end product, addition of isotonic agents, preservatives and/or buffers as required, and adjusting the pH value of the solution, e.g. using an acid, for example, hydrochloric acid, or a base, for example, aqueous sodium hydroxide, as needed. Finally, the volume of the solution may be adjusted with water to give the desired concentration of the ingredients.

A composition may be a stabilised formulation. The term “stabilised formulation” refers to a formulation with increased physical and/or chemical stability, preferably both. In general, a formulation must be stable during use and storage (in compliance with recommended use and storage conditions) until the expiration date is reached.

A solution or suspension may be made by dissolving an insulin-Fc conjugate of the invention in an aqueous medium.

### Methods of Therapy

The present invention relates to drugs for therapeutic use. More specifically the invention relates to the use of the insulin-Fc conjugate of the invention for the treatment or prevention of a metabolic disease or disorder or condition of a living animal body, including a human, which method comprises the step of administering to such a living animal body in need thereof, a therapeutically effective amount of an insulin-Fc conjugate according to the present invention.

In one embodiment, the invention provides a method for the treatment or alleviation of medical conditions relating to diabetes.

In another embodiment, the invention provides a method for the treatment or alleviation of a disease or disorder or condition of a living animal body, including a human, which disease, disorder or condition may be selected from a disease, disorder or condition relating to diabetes, Type 1 diabetes, Type 2 diabetes, impaired glucose tolerance, hyperglycemia, dyslipidemia, obesity, metabolic syndrome (metabolic syndrome X, insulin resistance syndrome), hypertension, cognitive disorders, atherosclerosis, myocardial infarction, stroke, cardiovascular disorders, coronary heart disease, inflammatory bowel syndrome, dyspepsia, or gastric ulcers, which method comprises administration to a subject in need thereof a therapeutically effective amount of the insulin-Fc conjugate of the invention.

In a third embodiment, the invention provides a method for the treatment or alleviation of a disease or disorder or condition of a living animal body, including a human, which disease, disorder or condition may be selected from a disease, disorder or condition relating to diabetes, Type 1 diabetes, Type 2 diabetes, impaired glucose tolerance, hyperglycemia, dyslipidemia, obesity, or metabolic syndrome (metabolic syndrome X, insulin resistance syndrome).

In a fourth embodiment, the invention provides a method for the treatment or alleviation of a disease or disorder or condition of a living animal body, including a human, which disease, disorder or condition may be selected from a disease, disorder or condition relating to diabetes, and in particular Type 1 diabetes, or Type 2 diabetes.

### PARTICULAR EMBODIMENTS

The invention is further described by the following non-limiting embodiments of the invention:

1. An insulin-Fc conjugate represented by Formula I:

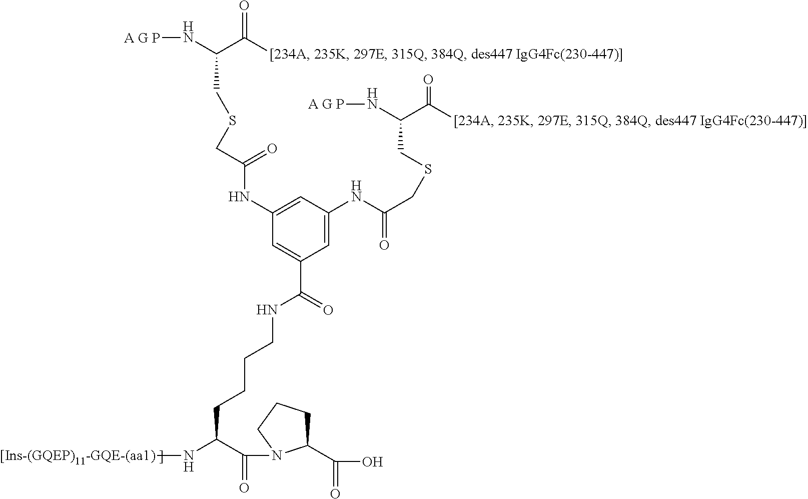

wherein Ins represents an analogue of human insulin, which insulin analogue is a two-chain insulin molecule comprising an A- and a B-chain, and which insulin analogue comprises the amino acid substitutions A14E, A21G, B25H, B29R, and the deletion desB30;

wherein (GQEP)11-GQE-(aa1) is a recombinant extension fused to the C-terminus of the insulin A-chain; and

wherein (aa1) is absent or proline (P).

2. The insulin-Fc conjugate according to embodiment 1, wherein (aa1) is absent.

3. The insulin-Fc conjugate according to embodiment 1, wherein (aa1) is proline (P).

4. The insulin-Fc conjugate according to any one of embodiments 1-3, wherein Ins is the insulin analogue [A14E, A21G, B25H, B29R, desB30] human insulin.

5. The insulin-Fc conjugate according to any one of embodiments 1-3, wherein Ins represents an insulin analogue further comprising the amino acid substitution B16H or the amino acid substitution B16E.

6. The insulin-Fc conjugate of embodiment 5, wherein Ins represents an insulin analogue selected from [A14E, A21G, B25H, B29R, desB30] human insulin, [A14E, A21G, B16H, B25H, B29R, desB30] human insulin, and [A14E, A21G, B16E, B25H, B29R, desB30] human insulin.

7. The insulin-Fc conjugate of embodiment 6, wherein Ins represents an insulin analogue further comprising the B16H substitution.

8. The insulin-Fc conjugate of embodiment 7, wherein Ins is the insulin analogue [A14E, A21G, B16H, B25H, B29R, desB30] human insulin.

9. The insulin-Fc conjugate of embodiment 6, wherein Ins represents an insulin analogue further comprising the B16E substitution.

10. The insulin-Fc conjugate of embodiment 9, wherein Ins is the insulin analogue [A14E, A21G, B16E, B25H, B29R, desB30] human insulin.

11. The insulin-Fc conjugate of embodiment 1, wherein the oligomer extended insulin-Fc conjugate is selected from

(A14E, A21G, A22(GQEP)₁₂, A70K\hat{ }, A71P, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl conjugate;

(A14E, A21G, A22(GQEP)₁₁, A66G, A67Q, A68E, A69K\hat{ }, A70P, B16H, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl conjugate; and

A14E, A21G, A22(GQEP)₁₁, A66G, A67Q, A68E, A69K\hat{ }, A70P, B16E, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl conjugate.

12. The insulin-Fc conjugate of embodiment 1, wherein the oligomer extended insulin-Fc conjugate is (A14E, A21G, A22(GQEP)₁₂, A70K\hat{ }, A71P, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl conjugate

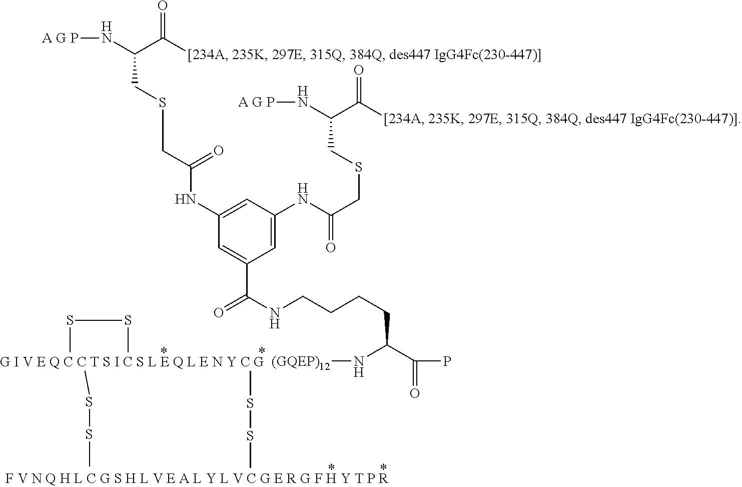

13. The insulin-Fc conjugate of embodiment 1, wherein the oligomer extended insulin-Fc conjugate is (A14E, A21G, A22(GQEP)₁₁, A66G, A67Q, A68E, A69K\hat{ }, A70P, B16H, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl conjugate

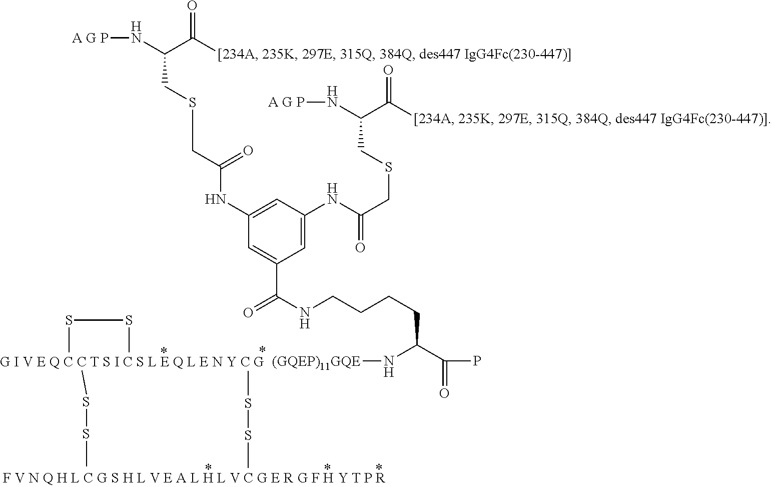

14. The insulin-Fc conjugate of embodiment 1, wherein the oligomer extended insulin-Fc conjugate is (A14E, A21G, A22(GQEP)₁₁, A66G, A67Q, A68E, A69K\hat{ }, A70P, B16E, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl conjugate

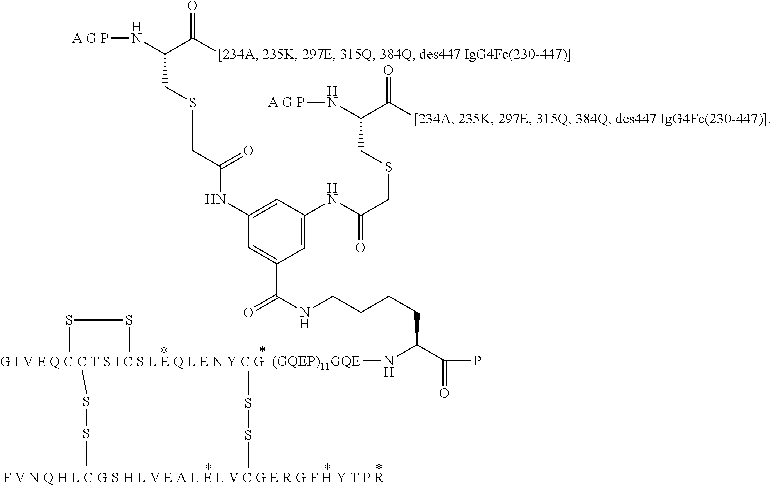

15. An intermediate compound Ins-(GQEP)₁₁-GQE-(aa1)-KP,

wherein Ins represents an analogue of human insulin, which insulin analogue is a two-chain insulin molecule comprising an A- and a B-chain, and which insulin analogue comprises the amino acid substitutions A14E, A21G, B25H, B29R, and the deletion desB30;

wherein (GQEP)11-GQE-(aa1) is a recombinant extension fused to the C-terminus of the insulin A-chain; and

wherein (aa1) is absent or proline (P).

16. The intermediate compound of embodiment 15, wherein (aa1) is absent.

17. The intermediate compound of embodiment 15, wherein (aa1) is proline (P).

18. The intermediate compound of any one of embodiments 15-17, wherein the insulin analogue further comprises the amino acid substitution B16H or B16E.

19. The intermediate compound of any one of embodiments 15-18, wherein the intermediate compound is selected from the group of

A14E, A21G, A22(GQEP)₁₂, A70K, A71P, B25H, B29R, desB30 human insulin (compound of Example 1.1);

A14E, A21G, A22(GQEP)₁₁, A66G, A67Q, A68E, A69K, A70P, B16H, B25H, B29R, desB30 human insulin (compound of Example 1.2); and

A14E, A21G, A22(GQEP)₁₁, A66G, A67Q, A68E, A69K, A70P, B16E, B25H, B29R, desB30 human insulin (compound of Example 1.3).

20. An intermediate compound of Formula II:

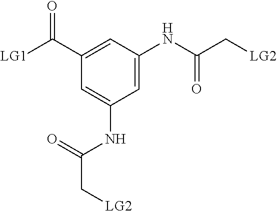

wherein

LG1 represents a leaving group reactive towards primary amino groups; and

LG2 represents the leaving group of a thiol reactive group.

21. The intermediate compound of embodiment 20, wherein the intermediate compound is (2,5-dioxopyrrolidin-1-yl)-3,5-bis[(2-bromoacetyl)amino]benzoate

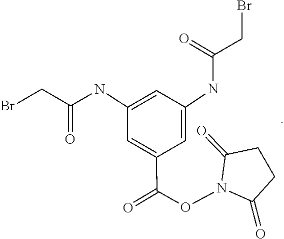

22. An intermediate compound of Formula III:

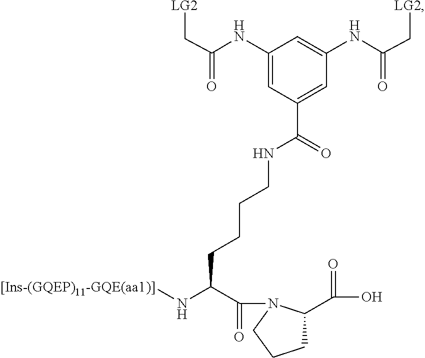

wherein Ins represents an analogue of human insulin, which insulin analogue is a two-chain insulin molecule comprising an A- and a B-chain, and which insulin analogue comprises the amino acid substitutions A14E, A21G, B25H, B29R, and the deletion desB30;

wherein (GQEP)11-GQE-(aa1) is a recombinant extension fused to the C-terminus of the insulin A-chain;

wherein (aa1) is absent or proline (P); and

wherein each LG2 represents the leaving group of a thiol reactive group.

23. The intermediate compound of embodiment 22, wherein the insulin analogue further comprises the amino acid substitution B16H or B16E.

24. The intermediate compound of any one of embodiments 22-23, wherein each LG2 represents Br.

25. The intermediate compound of embodiment 22, wherein the intermediate compound is selected from the group of

A14E, A21G, A22(GQEP)₁₂, A70K(N(eps)-3,5-bis[(2-bromoacetyl)amino]benzoyl), A71P, B25H, B29R, desB30 human insulin

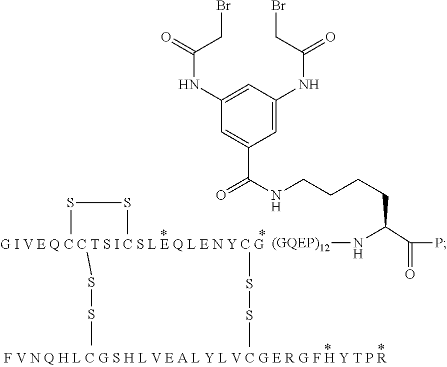

A14E, A21G, A22(GQEP)₁₁, A66G, A67Q, A68E, A69K(N(eps)-3,5-bis[(2-bromoacetyl)amino]benzoyl), A70P, B16H, B25H, B29R, desB30 human insulin

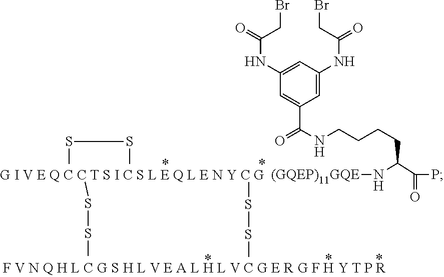

and

A14E, A21G, A22(GQEP)₁₁, A66G, A67Q, A68E, A69K(N(eps)-3,5-bis[(2-bromoacetyl)amino]benzoyl), A70P, B16E, B25H, B29R, desB30 human insulin

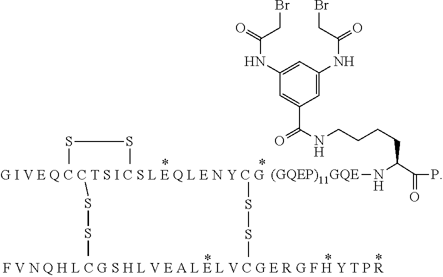

26. An intermediate compound which is 226A, 227G, 228P, 234A, 235K, 297E, 315Q, 384Q, des447 hIgG4-Fc(226-447).

27. A pharmaceutical composition comprising the insulin-Fc conjugate according to any one of embodiments 1-14, and one or more pharmaceutically acceptable carriers or diluents.

28. An insulin-Fc conjugate according to any of embodiments 1-14 for use as a medicament.

29. An insulin-Fc conjugate according to any of embodiments 1-14 for use in the treatment or alleviation of a disease or disorder or condition of a living animal body, including a human, which disease, disorder or condition may be selected from a disease, disorder or condition relating to diabetes, Type 1 diabetes, Type 2 diabetes, impaired glucose tolerance, hyperglycemia, dyslipidemia, obesity, metabolic syndrome (metabolic syndrome X, insulin resistance syndrome), hypertension, cognitive disorders, atherosclerosis, myocardial infarction, stroke, cardiovascular disorders, coronary heart disease, inflammatory bowel syndrome, dyspepsia, or gastric ulcers.

30. A method of treatment, prevention or alleviation of a metabolic disease or disorder or condition of a living animal body, including a human, which method comprises the step of administering to such a living animal body in need thereof, a therapeutically effective amount of the insulin-Fc conjugate according to any one of embodiments 1-14.

### EXAMPLES

The invention is further illustrated with reference to the following examples, which are not intended to be in any way limiting to the scope of the invention as claimed.

The following examples and general procedures refer to intermediate compounds and final products identified in the specification and in the synthesis schemes. The preparation of the compounds of the present invention is described in detail using the following examples, but the chemical reactions described are disclosed in terms of their general applicability to the preparation of compounds of the invention.

Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, i.e. by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials.

All temperatures are set forth in degrees Celsius and unless otherwise indicated, all parts and percentages are by weight when referring to yields and all parts are by volume when referring to solvents and eluents.

### Materials and Methods

### List of Abbreviations

AOC: Area over the curve
AUC: Are under the curve
BG; Blood glucose
BHK: Baby hamster kidney
BSPP: Bis(p-sulfonatophenyl)phenylphosphine dihydrate dipotassium salt
CV: Colum volume
DMF: Dimethylformaide
DTT: Dithiothreitol
ECD: Ectodomaine
EDC: N-ethyl-N′-dimethylaminopropyl carbodiimide
EDTA: Ethylenediaminetetraacetic acid
DIC: Diisopropylcarbodiimide
EA: Ethanolamine
EtOH: Ethanol
HEPES2-[4-(2-hydroxyethyl)piperazin-1-yl]ethanesulfonic acid
HI: Human insulin
HMWP: High molecular weight products
HAS: Humans serum albumin
IGF-1R: Insulin growth factor 1 receptor
IR: Insulin receptor
IR-A: Insulin receptor isotype A
IR-B: Insulin receptor isotype B
LCMS: Liquid Chromatography Mass Spectroscopy
LLOQ: Lower level of quantification
MeCN: Acetonitrile
MQ: MiliQ water
NMP: N-Methylpyrrolidone
OSu: N-Hydroxysuccinimidyl
PBS: Phosphate buffer saline
PD: Pharmacodynamics
PK: Pharmacokinetics
Rt: Retention time
RT: Room temperature
RP: Reverse phase
S.c.: Subcutaneously
SP: Sulfo-propyl
SPA: Scintillation proximity assay
STZ: Streptozotocin
TCEP: Tris(2-carboxyethyl)phosphine
TIV: Total ion count
TFA: Trifluoroacetic acid
Tris: tris(Hydroxymethyl)aminomethane or 2-amino-2-hydroxymethylpropane-1,3-diol
WGA: Wheat germ agglutinate
UPLC: Ultra Performance Liquid Chromatography

### General Methods of Detection and Characterisation

LCMS Method 1

System: Agilent 1290 infinity series

UPLC Column: Phenomenex Aeris widepore 3.6μ C4 50×2.1 mm

Detector: Agilent Technologies LC/MSD TOF 6230 (G6230A)

Detector setup: Ionisation method: Agilent Jet Stream source Scanning range: m/z min. 100, m/z max. 3200 linear reflector mode positive mode

Conditions: Step gradient: 5% to 90% B Gradient run-time: 10 minutes: 0-1 min 5-20% B, 1-7 min 20-90% B, 7-8 min 90% B 8-8.5 min 90-5% B 8.5-10 min 5% B

Flow rate: 0.40 ml/min fixed Column temperature: 40° C.

Eluents: Solvent A: 99.90% H2O, 0.02% TFA Solvent B: 99.90% CH3CN, 0.02% TFA Solvent C: NA

LCMS Method 2

System: Waters Acquity UPLC H-Class SQD2 2000

Column: Acquity UPLC BEH 1.7μ C18 100 Å 2,1×50 mm. Part no: 186002350

Detector: UV: PDA, SQD 2000

Detector setup: Ionisation method: ES+ Scanning range: 500-2000 Cone Voltage:

60 V Scantime: 0.5

Conditions: Linear gradient: 10% to 80% B Gradient run-time: 2.50 min Total run-time: 4 min Flow rate: 0.3 ml/min (0-2.51 min) and 0.8 ml/min (2.51-4.00 min)

Column temperature: 40° C. PDA: 210-400 nm Eluents: Solvent A: 99.90% H₂O, 0.1% TFA Solvent B: 99.90% CH₃CN, 0.1% TFA

Solvent C: NA

LCMS Method 3

LC-system: Waters Acquity UPLC H Class

Column: Waters Acquity BEH, C-18, 1.7 μm, 2.1 mm×50 mm

Detector: Waters Xevo G2-XS QTof

Detector setup: Ionisation method: ES Scanning range: 50-4000 amu

Operating mode: MS resolution mode positive/ne: positive mode Voltage: Capillary 3.00 kV; Sample cone 80 V, Source 60 V Temperature: Source 150° C., Desolvation 500° C., Scantime 0.500 s Interscandelay: 0.014 s

Conditions: Linear gradient: 5% to 95% B Gradient run-time: 4.0 minutes Total run-time: 7.0 minutes Flow rate: 0.4 ml/min Column temperature: 40° C.

Eluents: Solvent A: 99.90% MQ-water, 0.1% formic acid Solvent B: 99.90% acetonitrile, 0.1% formic acid Solvent C: 99.99% MQ water 0.01% TFA

Gradient: A 90-0%; B 5-95%, C 5%

Results specification and validation: Mass found of the compound is M/z, which is the molecular ion found ((M+z)/z) of the compound. Calculated Mass is the molecular weight of the desired compound. Calculated M/z is the molecular weight (M+z)/z of the desired compound. Purity: Total ion current (TIC) AUC of analyte peak, in percent of total AUC excl solvent peak, as reported by system software. Identity: Mass of each analyte mass peak expressed as m/z from highest to lowest. Scanning range is the range scanned in the method used. Detection method is e.g. linear reflector.

### Example 1: Preparation of the Oligomer Extended Insulin Compounds of the Invention

The oligomer extended fusion insulin compounds for use according to the invention may be produced by various techniques known in the art, e.g. as described in WO 2016/193380 A1.

### Example 1.1: Preparation of A14E, A21G, A22(GQEP)₁₂, A70K, A71P, B25H, B29R, desB30 Human Insulin

The title compound is the insulin analogue A14E, A21G, B25H, B29R, desB30 human insulin having a C-terminal A-chain extension of (GQEP)₁₂—KP (A-chain of SEQ ID NO:10, B-chain of SEQ ID NO:4).

Insulin-coding DNA was fused with DNA coding for the recombinant extensions. The DNA was cloned in yeast and the insulin was expressed and harvested. The extended insulin was expressed as single-chain precursor which was cleaved to two-chain extended insulin using trypsin.

Capture of the Precursor on SP Sepharose:

The SP column (approx. 200 mL) was regenerated with 0.5M NaOH and equilibrated with 0.1M citric acid pH 3.5. The capture run (cation exchange) was conducted at 20° C. The yeast supernatant was diluted with water 1:1 and loaded with a flow of 10-20 mL/min. A wash with 0.1 M citric acid pH 3.5 and a wash with 60% EtOH was performed. The analogue was eluted with 0.2 M Na-Acetate pH 5.5/40% EtOH. The SP-pool (appr. 600 ml) was diluted twice with water and 50 mM Glycine was added. pH was adjusted to 9.3, and 3-5 mg Trypsin per g insulin was added. Reaction was followed on the UPLC. After 3 hours, citric acid was added, pH adjusted to 3.5 and purified on a 50 mm 15p Gemini column. Column: 10 μm Gemini C18 50×250 mm 200 Å, 477 ml

Buffers: A: 10 ml formic acid/5 L 10% w/w acetonitrile

B: 70% w/w acetonitrile

Gradient: 10-50% B-buffer.

Gradient time: 120 min.

Flow: 80 ml/min.

Intact mass determined by LCMS (using LCMS Method 1):

Calculated average mass 10795.0; Found average mass 10796.0

### Example 1.2: Preparation of A14E, A21G, A22(GQEP)₁₁, A66G, A67Q, A68E, A69K, A70P, B16H, B25H, B29R, desB30 Human Insulin

The title compound is the insulin analogue A14E, A21G, B16H, B25H, B29R, desB30 human insulin having a C-terminal A-chain extension of (GQEP)₁₁-GQEKP.

A14E, A21G, A22(GQEP)₁₁, A66G, A67Q, A68E, A69K, A70P, B16H, B25H, B29R, desB30 human insulin (A-chain of SEQ ID NO:11, B-chain of SEQ ID NO:5) was prepared following the procedure described in Example 1.1.

### Intact Mass Determined by LCMS (Using LCMS Method 1):

Calculated average mass 10672.0; Found average mass 10673.0

### Example 1.3: Preparation of A14E, A21G, A22(GQEP)₁₁, A66G, A67Q, A68E, A69K, A70P, B16E, B25H, B29R, desB30 Human Insulin

The title compound is the insulin analogue A14E, A21G, B16E, B25H, B29R, desB30 human insulin having a C-terminal A-chain extension of (GQEP)₁₁-GQEKP.

A14E, A21G, A22(GQEP)₁₁, A66G, A67Q, A68E, A69K, A70P, B16E, B25H, B29R, desB30 human insulin (A-chain of SEQ ID NO:11, B-chain of SEQ ID NO:6) was prepared following the procedure described in Example 1.1.

### Intact Mass Determined by LCMS (Using LCMS Method 1):

Calculated average mass 10664.4; Found average mass 10665.0

### Example 2: Preparation of the Fc Component

### Preparation of 226A, 227G, 228P, 234A, 235K, 297E, 315Q, 384Q, des447 hIgG4-Fc(226-447)

The DNA sequence encoding MAGP-IgG4 Fc amino acid sequence (225M, 226A, 227G, 228P, 234A, 235K, 297E, 315Q, 384Q, des447, IgG4-Fc(226-447)) amino acid sequence was inserted into modified vector (pET-11 based) under the control of T7 promoter and transformed into a BL21 (DE3) derived host strain

(AGPCPAPEAKGGPSVFLFPPKPKDTLMISRTPEVTCVVVDVSQEDPEV
QFNWYVDGVEVHNAKTKPREEQFESTYRVVSVLTVLHQDWLQGKEYKCK
VSNKGLPSSIEKTISKAKGQPREPQVYTLPPSQEEMTKNQVSLTCLVKG
FYPSDIAVEWESQGQPENNYKTTPPVLDSDGSFFLYSRLTVDKSRWQEG
NVFSCSVMHEALHNHYTQKSLSLSLG)

226A, 227G, 228P, 234A, 235K, 297E, 315Q, 384Q, des447 hIgG4-Fc(226-447) (SEQ ID NO:7) was then produced at high level from E. coli as inclusion bodies. The inclusion bodies were washed two times using distilled water, and solubilized in 6M Urea, 10 mM DTT, 50 mM Tris pH 9.0 at 10 mg/ml. The solubilized inclusion bodies were fast diluted to the refolding solution (20 mM EA pH 10.0, 3.3M Urea, 0.125 mM Cysteine 0.125 mM Cystine pH 8.5) to a final concentration at 1 mg/mL overnight. The refolded protein was captured by using AIEX Q Sepharose Fast Flow column

Buffers: A: 20 mM Tris, pH 8.5, B: 20 mM Tris, 500 mM NaCl, pH 8.5 Gradient: 15-30%, 15 CV

The second column anion exchange Source 30Q

Buffers: A: 20 mM His, pH 6.2, B: 20 mM His, 100 mM NaCl, pH 8.5

Gradient: 10-40%, 20 CV

The purified Fc molecule were finally solubilized in 10 mM Tris pH7.5, 30 mM NaCl.

### Intact Mass Determined by LCMS (Using LCMS Method 1):

Calculated average mass 49583.3; Found average mass 49585.0

### Example 3: Preparation of the Linker for Derivatisation of Insulin Analogues

### Synthesis of (2,5-dioxopyrrolidin-1-yl)-3,5-bis[(2-bromoacetyl)amino]benzoate

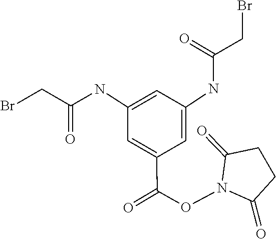

3,5-diaminobenzoic acid (0.5 g) was dissolved in 5 ml anhydrous DMF. The reaction was cooled on ice while bromoacetic acid anhydride (1.8 g) dissolved in DMF (1.8 mL) was added drop-wise at +5° C. in the reaction. The ice bath was removed and the reaction was stirred for 4 hours at RT. To the reaction was added 50 ml ice cold water, a grey precipitate was formed. The mixture was stored in the fridge overnight. The precipitate was filtered off and washed with water. The precipitate was dissolved in 2-methy-tetrahydrofuran (20 mL), dried over Na₂SO₄, filtered, and concentrated in vacuo. To the residue was added N-hydroxysuccinimide (416 mg) and DIC (1.0 mL). After stirring at RT for 3 hours, the mixture was concentrated in vacuo. Acetonitrile (20 mL) was added, and urea was filtered off. The filtrate was reduced to ˜5 mL in vacuo. Precipitation from diethylether. The precipitate was washed and centrifuged twice. The isolated compound was dried under a stream of nitrogen in vacuum.

LCMS Method 3: m/1: calculated 492.1; found 491.9.

### Example 4: Preparation of Insulin Derivatives

The preparation of a representative insulin derivative comprising the oligomer extended insulin analogue and a linking group is given in Example 4.1. The insulin derivatives of Examples 4.2-4.3 are prepared by the method provided in Example 4.1, unless otherwise stated.

### Example 4.1: Synthesis of A14E, A21G, A22(GQEP)₁₂, A70K(N(eps)-3,5-bis[(2-bromoacetyl)amino]benzoyl), A71P, B25H, B29R, desB30 Human Insulin

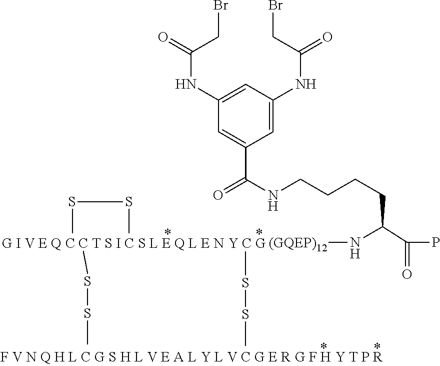

A solution of A14E, A21G, A22(GQEP)₁₂, A70K, A71P, B25H, B29R, desB30 human insulin (Example 1.1) (400 mg) in 0.1M Na₂CO₃ (10 ml) and acetonitrile (2 ml) was adjusted to pH 11.1 with 1N NaOH. (2,5-dioxopyrrolidin-1-yl)-3,5-bis[(2-bromoacetyl)amino]benzoate (45 mg) of Example 3 dissolved in NMP (0.5 ml), was added drop-wise under vigorous stirring. pH was readjusted to 11.1. After 20 min, additional (2,5-dioxopyrrolidin-1-yl)-3,5-bis[(2-bromoacetyl)amino]benzoate (18 mg) in NMP (0.5 ml) was added. After 40 min, pH was adjusted with TFA to 2.0 and water was added up to 40 ml. Purification by RP-chromatography.

Column: Phenomenex, Gemini-NX, AXIA, 5μ, C18, 110 Å, 30×250 mm

Buffers: A: 0.1% TFA in water; B: 0.1% TFA in acetonitrile

Gradient: 20-50% B over 40 min

The product pool was lyophilized to give the title compound in 27% yield.

LCMS Method 2: Calc. mass 11171.5; Found mass: 11170.0, Rt 1.31 min

### Example 4.2: Synthesis of A14E, A21G, A22(GQEP)₁₁, A66G, A67Q, A68E, A69K(N(eps)-3,5-bis[(2-bromoacetyl)amino]benzoyl), A70P, B16H, B25H, B29R, desB30 Human Insulin

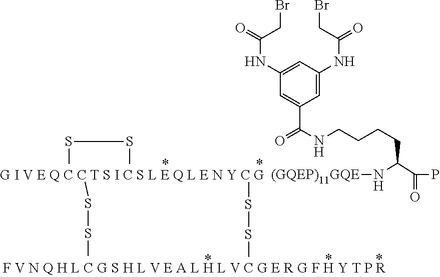

The title compound was prepared from of A14E, A21G, A22(GQEP)₁₁, A66G, A67Q, A68E, A69K, A70P, B16H, B25H, B29R, desB30 human insulin (Example 1.2), and (2,5-dioxopyrrolidin-1-yl)-3,5-bis[(2-bromoacetyl)amino]benzoate of Example 3, following the general linker to insulin conjugation procedure described in Example 4.1.

LCMS Method 2: Calc. mass 11048.4; Found mass: 11048.0, Rt 1.26 min

### Example 4.3: Synthesis of A14E, A21G, A22(GQEP)₁₁, A66G, A67Q, A68E, A69K(N(eps)-3,5-bis[(2-bromoacetyl)amino]benzoyl), A70P, B16E, B25H, B29R, desB30 Human Insulin

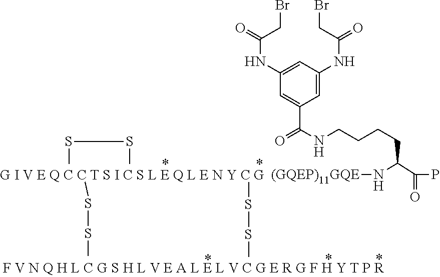

The title compound was prepared from of A14E, A21G, A22(GQEP)₁₁, A66G, A67Q, A68E, A69K, A70P, B16E, B25H, B29R, desB30 human insulin (Example 1.3), and (2,5-dioxopyrrolidin-1-yl)-3,5-bis[(2-bromoacetyl)amino]benzoate of Example 3, following the general linker to insulin conjugation procedure described in Example 4.1.

LCMS Method 2: Calc. mass 11040.4; Found mass: 11041.0, Rt 1.31 min

### Example 5: Preparation of Insulin-Fc Conjugates

The preparation of a representative insulin-Fc conjugate is given in Example 5.1. The insulin conjugates of Examples 5.2-5.3 were prepared by the method provided in Example 5.1, unless otherwise stated.

### Example 5.1: Preparation of (A14E, A21G, A22(GQEP)₁₂, A70K\hat{ }, A71P, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl Conjugate

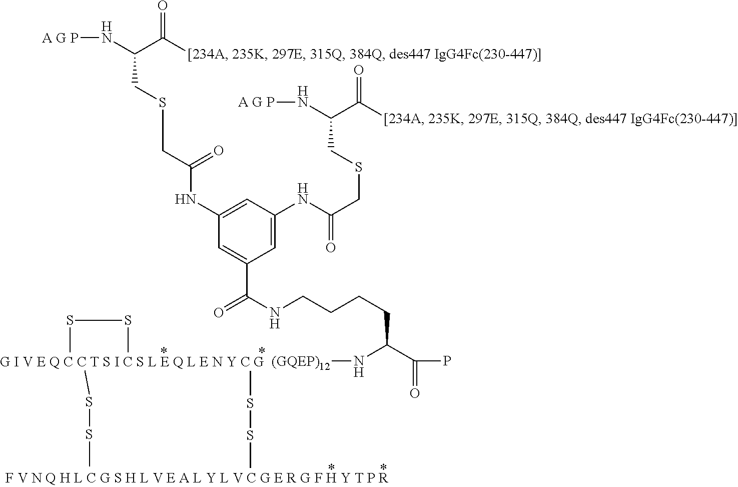

To 226A, 227G, 228P, 234A, 235K, 297E, 315Q, 384Q, des447 hIgG4-Fc(226-447) of Example 2 (441 mg) 7.35 mg/ml in 20 mM Tris, 30 mM NaCl, pH 7.5 (60 ml) was added EDTA (125 mg). BSPP (38 mg), dissolved in water (1 ml) was added and stirred gently at RT for 18 hours. The mixture was buffer exchanged to 20 mM Tris, 10 mM EDTA pH 7.5 using a 412 ml G-25 fine sephadex desalting column. To the elution pool (95 ml) at pH 7.6 was added A14E, A21G, A22(GQEP)₁₂, A70K(N(eps)-3,5-bis[(2-bromoacetyl)amino]benzoyl), A71P, B25H, B29R, desB30 human insulin of Example 4.1 (100 mg) dissolved in water (5 ml) and acetonitrile (1 ml) drop-wise at RT. pH was adjusted to 7.6 with 1N NaOH and stirred at RT overnight. The reaction was diluted with water (100 ml), pH adjusted to 8.9 with 1N NaOH, conductivity 3.05 mS/cm and was purified by anion exchange.

A buffer: 20 mM Tris at pH 9.0 (1.5 mS/cm)

B buffer: 20 mM Tris, 500 mM NaCl at pH 9.0 (43 mS/cm)

Flow: 35 ml/min

Gradient, step: 0-30% B over 1CV, 30% B 1 CV, 30-70% B over 4 CV, 70-90% B over 3 CV The compound pool was buffer exchanged to water followed by lyophylization.

Column: Desalting column, 400 ml Sephadex G-25 fine

The product pool was lyophilized. Yield 43%.

LCMS Method 1: Calc. mass: 60595.0; Found mass: 60596.3

### Example 5.2: Preparation of the (A14E, A21G, A22(GQEP)₁₁, A66G, A67Q, A68E, A69K\hat{ }, A70P, B16H, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl Conjugate

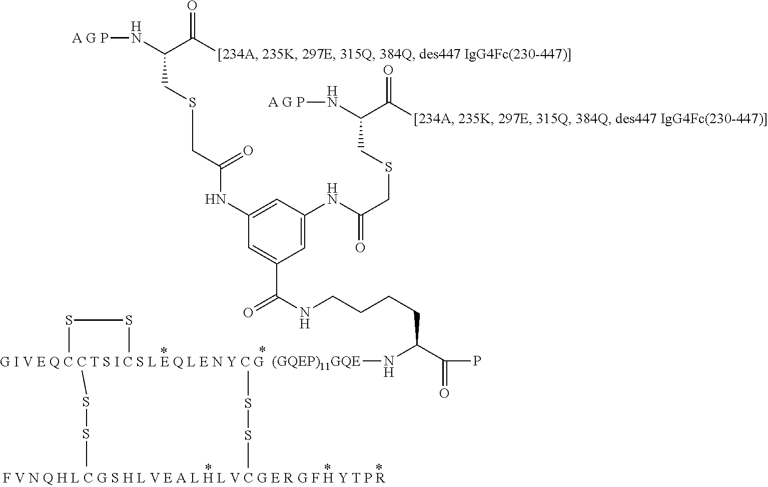

The title compound was synthesised from A14E, A21G, A22(GQEP)₁₁, A66G, A67Q, A68E, A69K(N(eps)-3,5-bis[(2-bromoacetyl)amino]benzoyl), A70P, B16H, B25H, B29R, desB30 human insulin of Example 4.2, and 226A, 227G, 228P, 234A, 235K, 297E, 315Q, 384Q, des447 hIgG4-Fc(226-447) of Example 2, following the general insulin Fc conjugation procedure described in Example 5.1.

LCMS Method 1: Calc. mass: 60471.9; Found mass: 60473.1

### Example 5.3: Preparation of (A14E, A21G, A22(GQEP)₁₁, A66G, A67Q, A68E, A69K\hat{ }, A70P, B16E, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl Conjugate

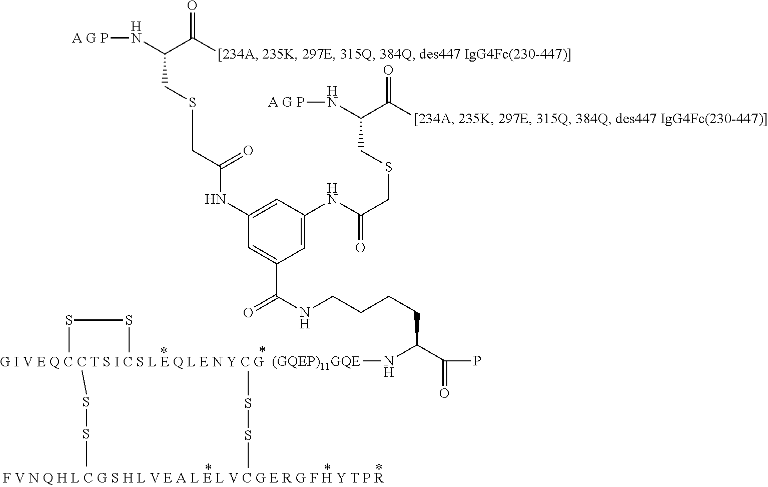

The title compound was synthesised from A14E, A21G, A22(GQEP)₁₁, A66G, A67Q, A68E, A69K(N(eps)-3,5-bis[(2-bromoacetyl)amino]benzoyl), A70P, B16E, B25H, B29R, desB30 human insulin of Example 4.3, and 226A, 227G, 228P, 234A, 235K, 297E, 315Q, 384Q, des447 hIgG4-Fc(226-447) of Example 2, following the general insulin Fc conjugation procedure described in Example 5.1.

LCMS Method 1: Calc. mass: 60463.8; Found mass: 60465.9

### Example 6: Insulin Receptor Affinity Measured on Solubilised Receptors

The relative binding affinity of the insulin analogues of the invention for the human insulin receptor (IR) is determined by competition binding in a scintillation proximity assay (SPA) (according to Glendorf T et al. (2008) Biochemistry 47, 4743-4751), see results in Table 1. The affinities are reported relative to affinity of human insulin for insulin receptor A (100%).

In brief, dilution series of a human insulin standard and the insulin analogue to be tested are performed in 96-well Optiplates (Perkin-Elmer Life Sciences) followed by the addition of [¹²⁵I-A14Y]-human insulin, anti-IR mouse antibody 83-7, solubilised human IR-A (semipurified by wheat germ agglutinin chromatography from baby hamster 15 kidney (BHK) cells overexpressing the IR-A holoreceptor), and SPA beads (Anti-Mouse polyvinyltoluene SPA Beads, GE Healthcare) in binding buffer consisting of 100 mM HEPES (pH 7.8), 100 mM NaCl, 10 mM MgSO4, and 0.025% (v/v) Tween 20. Plates are incubated with gentle shaking for 22-24 h at 22° C., centrifuged at 2000 rpm for 2 minutes and counted on a TopCount NXT (Perkin-Elmer Life Sciences).

Data from the SPA are analysed according to the four-parameter logistic model (Volund A (1978) Biometrics 34 357-365), and the binding affinities of the analogues calculated relative to that of the human insulin standard measured within the same plate.

TABLE 1
In vitro data

| | Insulin receptor affinity |
|---|---|
| Compound of | [IC50 relative to HI (%)] |
| Example No. | Mean Value |
| 5.1 | 4.5 |
| 5.2 | 1.6 |
| 5.3 | 0.4 |

The data show that all of the tested compounds of the invention bind to the insulin receptor in the range 0.4 to 4.5% relative to human insulin. These affinities are considered adequate to facilitate blood glucose lowering following in vivo administration.

### Example 7: Insulin and Insulin-Like Growth Factor-1 Receptor Affinities Measured on Membrane Associated Receptors

Membrane-associated human IR and IGF-1R are purified from BHK cells stably transfected with the pZem219B vector containing either the human IR-A, IR-B or IGF-IR insert. BHK cells are harvested and homogenized in ice-cold buffer (25 mM HEPES pH 7.4, 25 mM CaCl₂ and 1 mM MgCl₂, 250 mg/L bacitracin, 0.1 mM Pefablock). The homogenates are layered on a 41% (w/v) sucrose cushion and centrifuged for 75 minutes at 95000 g at 4° C. The plasma membranes are collected, diluted 1:5 with buffer (as above) and centrifuged again for 45 minutes at 40000 g at 4° C. The pellets are resuspended in a minimal volume of buffer and drawn through a needle (size 23) three times before storage at −80° C. until usage. The relative binding affinity for either of the membrane-associated human IR-A, IR-B or IGF-1R is determined by competition binding in a SPA setup. IR assays are performed in duplicate in 96-well OptiPlates (Perkin-Elmer Life Sciences). Membrane protein is incubated with gentle agitation for 150 minutes at 25° C. with 50 pM [¹²⁵IA14Y]-human insulin in a total volume of 200 pL assay buffer (50 mM HEPES, 150 mM NaCl, 5 mM MgSO4, 0.01% Triton X-100, 0.1% (w/v) HSA (Sigma A1887), Complete EDTA-free protease inhibitors), 50 μg of wheat germ agglutinate (WGA)-coated PVT microspheres (GE Healthcare) and increasing concentrations of ligand. Assays are terminated by centrifugation of the plate at 2000 rpm for 2 minutes and bound radioactivity quantified by counting on a TopCount NXT (Perkin-Elmer Life Sciences).

IGF-1R assays are conducted essentially as for the IR binding assays except that membrane-associated IGF-1R and 50 pM [¹²⁵I-Tyr31]-human IGF-1 were employed. Data from the SPA are analysed according to the four-parameter logistic model (Vølund A (1978) Biometrics 34 357-365), and the binding affinities of the analogues to be tested are calculated relative to that of the human insulin standard measured within the same plate.

IR (A isoform), IR (B isoform), and IGF-1R binding data of the compounds of the invention are given in Table 2, below.

The data show that all of the tested compounds of the invention bind to the insulin receptor A and B in the range 0.6 to 3.1% relative to human insulin. These affinities are considered adequate to facilitate blood glucose lowering following in vivo administration. Furthermore the data show that the compounds of the invention binds with lower affinity to the IGF1R (0.3% to 0.9%) than to IR-A and IR-B.

TABLE 2
Insulin and Insulin-Like Growth factor-1 receptor affinities

| Compound of | hIR-A mem | hIR-B mem | IGF1R mem |
|---|---|---|---|
| Example no | Rel. aff. (%) | Rel. aff. (%) | Rel. aff. (%) |
| 5.1 | 2.97 | 3.05 | 0.91 |
| 5.2 | 1.27 | 1.26 | 0.10 |
| 5.3 | 0.66 | 0.59 | 0.29 |

### Example 8: Lipogenesis in Rat Adipocytes

As a measure of in vitro potency of the insulin conjugatess of the invention, lipogenesis can be used. Primary rat adipocytes are isolated from the epididymale fat pads and incubated with 3H-glucose in buffer containing e.g. 0.1% fat free HSA and either standard (human insulin, HI) or insulin conjugates of the invention. The labelled glucose is converted into extractable lipids in a dose dependent way, resulting in full dose response curves. The result is expressed as relative potency (%) with 95% confidence limits of insulin of the invention compared to standard (HI).

Data are given in Table 3 below.

The data show that all of the tested compounds of the invention have a potency on lipogenesis in the range 0.6 to 2.9% relative to human insulin. These affinities are considered adequate to facilitate blood glucose lowering following in vivo administration

TABLE 3
Lipogenesis in rat adipocytes

| Compound of | Lipogenesis |
|---|---|
| Example No | Rel. pot. (%) |
| 5.1 | 2.86 |
| 5.2 | 1.14 |
| 5.3 | 0.64 |

### Example 9: In Vivo PK/PD Studies

Insulin-Fc conjugates were tested in vivo by subcutaneous (s.c.) administration to normal and streptozotocin (STZ) treated male Sprague-Dawley (SD) rats. The normal rats had a body weight of approx. 300 g at the time of dosing. The STZ treated rats had a body weight of approx. 400 g at the time of dosing and were treated intravenously with 40 mg/kg STZ (Sigma-Aldrich, no. S0130) for induction of diabetes four days before dosing with the insulin-Fc conjugates.

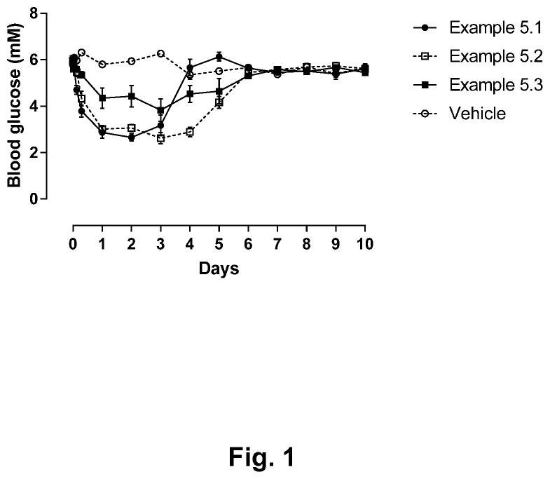

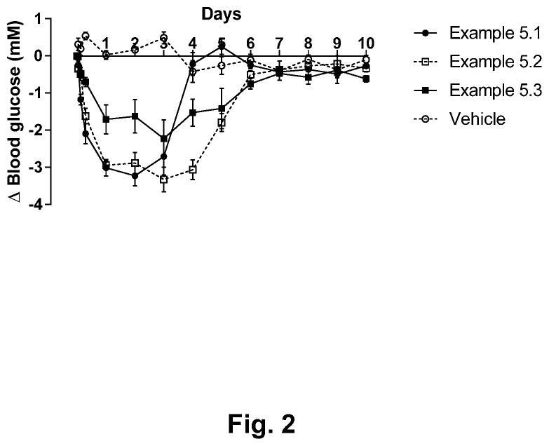

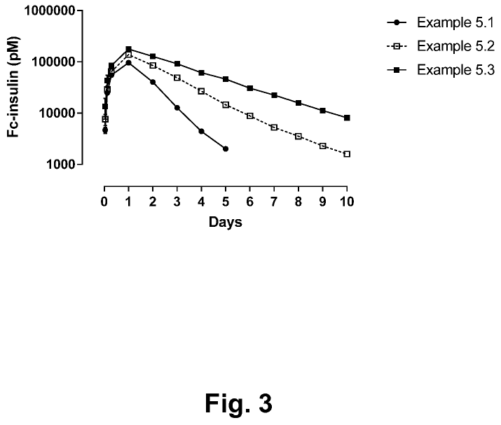

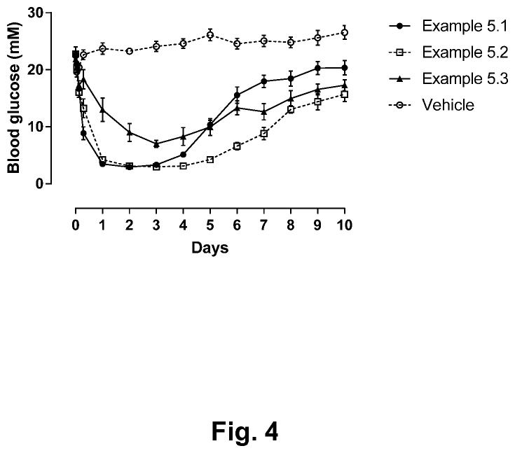

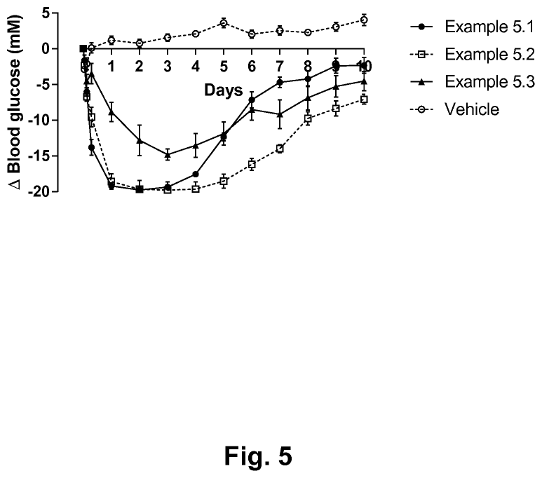

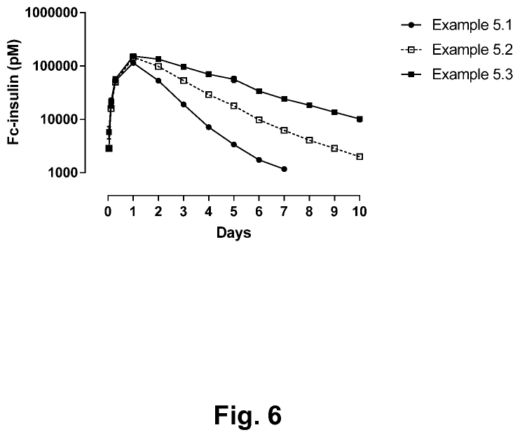

The rats were dosed s.c. in the neck region with a dose of 30 nmol/kg of the insulin-Fc conjugates. Blood samples for blood glucose and plasma insulin analysis were collected from the tongue vein up to 10 days post dosing. The insulin-Fc concentrations were measured by immuno-assay (LLOQ=1000 pM) and the resulting half-life values after s.c. dosing are shown in Table 4. The PK profiles for normal rats are shown in FIG. 3 and for streptozotocin treated rats in FIG. 6. Blood glucose (BG) was measured using Biosen S Line (EKF) and the BG and baseline corrected BG profiles for normal rats are shown in FIGS. 1 and 2 and for streptozotocin treated rats in FIGS. 4 and 5 respectively. In addition, the areas over the curve of the 10-day baseline corrected BG profiles (AOC_{BG}) are shown in Table 5.

TABLE 4
Pharmacokinetic data (half-life mean value) after
s.c. dosing of 30 nmol/kg of the insulin-Fc conjugates
to normal and streptozotocin treated rats.

| Compound of | PK normal rat | PK STZ rat |
|---|---|---|
| Example No. | [T_{1/2, s.c.} (h)] | [T_{1/2, s.c.} (h)] |
| 5.1 | 20 | 24 |
| 5.2 | 38 | 39 |
| 5.3 | 48 | 50 |

TABLE 5
Area over the curve of baseline corrected blood glucose profiles
up to 10 days post dosing (AOC_{BG}, mean value) given as difference
from vehicle after s.c. dosing of 30 nmol/kg of the insulin-
Fc conjugates to normal and streptozotocin treated rats.

| Compound of | AOC_{BG} normal rat | AOC_{BG} STZ rat |
|---|---|---|
| Example No. | [mM*hr] | [mM*hr] |
| 5.1 | 251 | 3181 |
| 5.2 | 364 | 4106 |
| 5.3 | 248 | 2771 |

The data shows that the tested compounds of the invention were very potently able to lower blood glucose over long time (4-7 days) in normal rats and up to at least 10 days in streptozotocin treated rats.
The data shows that the insulin-Fc conjugates of the invention all display very long PK profiles when administered to normal and streptozotocin treated rats.

### Example 10: Stability Studies

### Preparation of Samples for Stability Studies

Samples for stability studies were prepared by weighing out 8-10 mg freeze-dried material. The freeze-dried material was solubilized to 100 pM in a premade solution A (5 mM phophate (pH 7.4), 140 mM NaCl and 70 ppm polysorbate 20). Protein concentration was determined using absorbance at 280 nm using a Nanodrop 2000 (Thermo Fisher Scientific Inc.) and theoretical extinction coefficients. Dilution into solution A was done to reach a final concentration of 30 μM.

Purity by Anionic Exchange Chromatography

Anionic exchange chromatography was performed on an Ultra-Performance Liquid Chromatography system (Waters Acquity H-Class) equipped with a fluorescence detector (Waters Acquity FLR Detector). A Bio SAX column with dimensions 50×4.6 mm from Agilent, kept at 30° C., was used in gradient elution mode. Mobile phase A was 5/95 (v/v), acetonitrile/water with 25 mM BIS-TRIS propane, pH 7.3 and mobile phase B was 5/95 (v/v), acetonitrile/water with 25 mM BIS-TRIS propane and 350 mM NaCl, pH 7.3. Injection volume was 3 μL. The flow rate was kept at 0.5 mL/min with a linear gradient from 10 to 47% B in 3 min followed by a linear gradient from 47 to 55% B in 30 min. Peaks were detected with an emission wavelength of 331 nm and an excitation wavelength of 280 nm and data processing is performed with the EMPOWER 3 software from Waters. Samples were analysed for purity as percent API in relation to all other species.

Reported as Δ purity %=purity %(5° C.)−purity % (30° C.).

High-Molecular Weight Products by Native Size-Exclusion Chromatography

Native size-exclusion chromatography was performed on a High-Performance Liquid Chromatography system (Waters Alliance) equipped with a UV/Vis detector (Waters 2489 UV/Vis detector). A Waters XBridge BEH 200 Å, 3.5 μm column with dimensions 7.8×300 mm, kept at 30° C., was used. The mobile phase was 50 mM sodium phosphate (pH 7.0), 50 mM NaCl, 5% isopropyl alcohol (v/v). Injection volume was 5 μL. The flow rate was kept at 0.5 mL/min. Peaks were detected using absorbance at 215 nm and data processing is performed with the EMPOWER 3 software from Waters. Samples were analysed for high molecular weight products (HMWP) by relating the total integral of material eluting before main peak with main monomeric peak. In all chromatograms, full baseline separation was observed.

Reported as Δ HMWP %=HMWP %(5° C.)−HMWP % (30° C.).

Results from Stability Studies

Stability of Fc-insulin analogues was evaluated by comparing purity from anion exchange chromatography and formation of high molecular weight products (HMWP) at 5° C. (control) versus 30° C. (heat induced stress). The results are seen in Table 6. Purity was observed to be 3-7% lower in the stressed samples than the control. The amount of HMWP was decreased by 0.5-2% in the stressed sample versus the control, i.e. some aggregated material has disassociated during incubation at 30° C.

TABLE 6
Purity and HMWP of API measured with
anionic exchange chromatography

| Compound of | | |
|---|---|---|
| Example No. | Δ purity | Δ HMWP % |
| 5.1 | 3.3% | −2.17 |
| 5.2 | 6.7% | −0.63 |
| 5.3 | 4.3% | Missing* |

*Missing due to instrument error

### Example 11: SPR Analysis of the Binding of hFcRn ECD

The powders of the Insulin-Fc conjugates of Example 5.1, 5.2 and 5.3 were dissolved at 5 mg/ml using PBS (pH 7.4) (Sigma-Aldrich, catalogue #D8537) and then the binding affinities of the hFcRn ectodomain for the three Insulin-Fc conjugates samples at pH 6.0 and 25° C. were measured using a Biacore 4000 instrument.

First, the three Insulin-Fc samples diluted at 10 ug/ml and the wild-type hIgG4 Fc (produced in HEK293 cells, i.e. glycosylated) diluted at 30 ug/ml in 10 mM sodium acetate (pH 4.5) (GE Healthcare, catalogue #BR100350) were immobilized on a CMD50L sensor chip (XanTec Bioanalytics GmbH) at spot 2 or 4 at 70-90 RU in each used flow cell via amine coupling with 1×HBS-EP+ (pH 7.4) (GE Healthcare, catalogue #BR-1006-69) used as the running buffer. The wild-type hIgG4 Fc was used as the control sample. In each used flow cell, spots 2, 3 and 4 were activated by 100 mM N-hydroxysuccinimide (NHS) and 400 mM N-ethyl-N′-dimethylaminopropyl carbodiimide hydrochloride (EDC) mixed at equal volume before the immobilization and blocked by 1 M ethanolamine hydrochloride-NaOH (pH 8.5) after the immobilization. NHS, EDC and ethanolamine were obtained from the Amine Coupling Kit, type 2 (GE Healthcare, catalogue #BR100633).

Next, the hFcRn ectodomain diluted at 4000 nM with 2-fold serial dilutions was injected through each used flow cell for 60 sec to allow for binding of the hFcRn ectodomain to the Insulin-Fc conjugates and wild-type hIgG4 Fc samples immobilized on the sensor chip, followed by dissociation for 180 sec. 1×PBS-EP+ (pH 6.0) was used as the dilution buffer for the hFcRn ectodomain and the running buffer in each binding cycle at the flow rate of 30 ul/min. After each binding cycle, the sensor chip was regenerated with a 60 sec injection of 1×PBS-EP+ (pH 7.4) at the flow rate of 30 ul/min. The 1×PBS-EP+ (pH 7.4) buffer was prepared by diluting 0.5 M EDTA (pH 8.0) (Invitrogen, catalogue #15575038) at 3 mM into 1×PBS-P+ (GE Healthcare, catalogue #28995084). The 1×PBS-EP+ (pH 6.0) buffer was prepared by adjusting the pH of the 1×PBS-EP+ buffer from 7.4 to 6.0 using ˜10 M HCl. The affinities (equilibrium dissociation constants, K_{D}) were determined via steady-state fitting of the binding curves using the 1:1 binding model in Biacore 4000 Evaluation Software 1.0 (GE Healthcare).

The results are shown in Table 7. The results show that the binding to of the compounds of the invention is comparable to wild type hIgG4-Fc.

TABLE 7
SPR Analysis of the Binding of hFcRn ECD at pH 6.0

| Insulin Fc conjugate | Kₙ |
|---|---|
| of Example No | (μM) |
| 5.1 | 1.5 |
| 5.2 | 1.6 |
| 5.3 | 1.5 |
| WT hlgG4 Fc(227-447) | 1.5 |
| (SEQ ID NO: 12) | |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An insulin-Fc conjugate represented by Formula I: 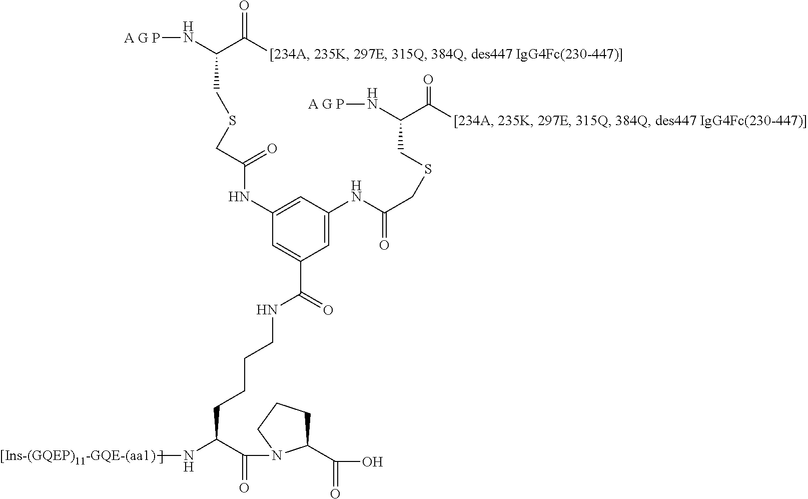
wherein Ins represents an analogue of human insulin, wherein said insulin analogue is a two-chain insulin molecule comprising an A- and a B-chain, and wherein said insulin analogue comprises the amino acid substitutions A14E, A21G, B25H, B29R, and the deletion desB30;
wherein (GQEP)₁₁-GQE-(aa1) is a recombinant extension fused to the C-terminus of the insulin A-chain; and
wherein (aa1) is absent or proline (P).

2. The insulin-Fc conjugate according to claim 1, wherein Ins represents an insulin analogue further comprising the amino acid substitution B16H or the amino acid substitution B16E.

3. The insulin-Fc conjugate of claim 1, wherein Ins represents an insulin analogue selected from [A14E, A21G, B25H, B29R, desB30] human insulin, [A14E, A21G, B16H, B25H, B29R, desB30] human insulin, and [A14E, A21G, B16E, B25H, B29R, desB30] human insulin.

4. The insulin-Fc conjugate of claim 1, wherein the oligomer extended insulin-Fc conjugate is selected from
(A14E, A21G, A22(GQEP)₁₂, A70K\hat{ }, A71P, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl conjugate 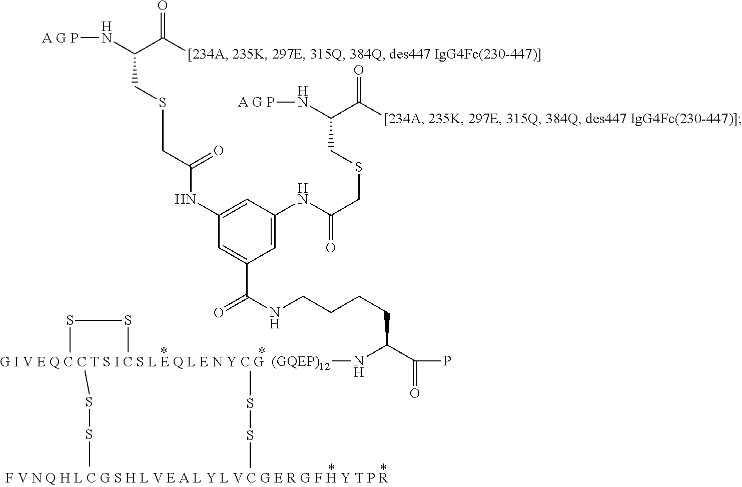
(A14E, A21G, A22(GQEP)₁₁, A66G, A67Q, A68E, A69K\hat{ }, A70P, B16H, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl conjugate 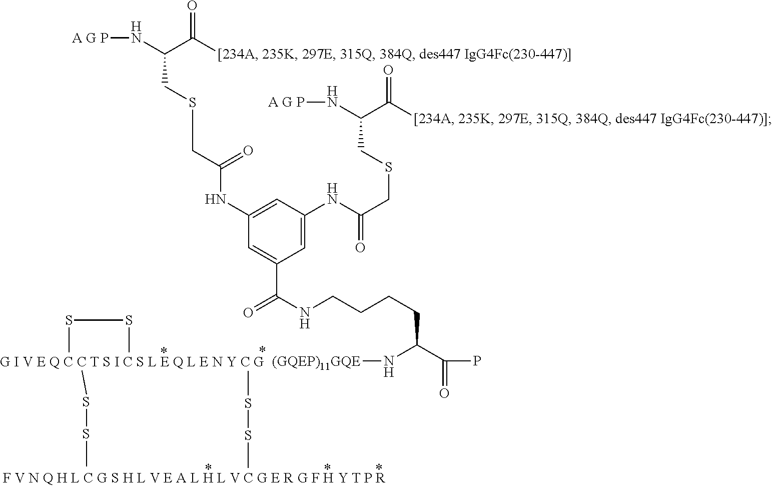
and
(A14E, A21G, A22(GQEP)₁₁, A66G, A67Q, A68E, A69K\hat{ }, A70P, B16E, B25H, B29R, desB30 human insulin)/(226A, 227G, 228P, 234A, 235K, 297E, 315Q, 384Q, des447 hIgG4-Fc(226-447))3,5-bis[(2-acetyl)amino]benzoyl conjugate

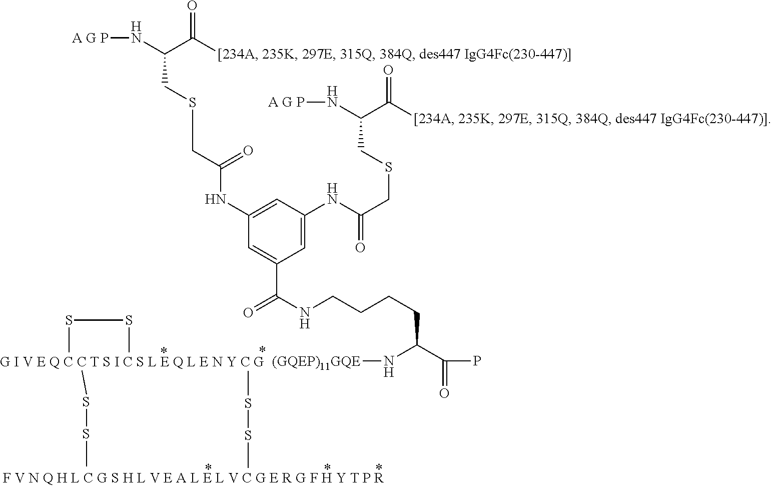

5. An intermediate compound Ins-(GQEP)₁₁-GQE-(aa1)-KP,
wherein Ins represents an analogue of human insulin, wherein said insulin analogue is a two-chain insulin molecule comprising an A- and a B-chain, and wherein said insulin analogue comprises the amino acid substitutions A14E, A21G, B25H, B29R, and the deletion desB30;
wherein (GQEP)₁₁-GQE-(aa1) is a recombinant extension fused to the C-terminus of the insulin A-chain; and
wherein (aa1) is absent or proline (P).

6. The intermediate compound of claim 5, wherein the insulin analogue further comprises the amino acid substitution B16H or B16E.

7. An intermediate compound of Formula II: 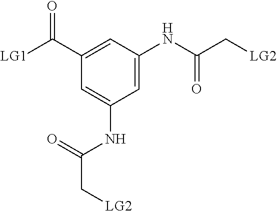
wherein
LG1 represents a leaving group reactive towards primary amino groups; and
LG2 represents the leaving group of a thiol reactive group.

8. The intermediate compound of claim 7, wherein the intermediate compound is (2,5-dioxopyrrolidin-1-yl)-3,5-bis[(2-bromoacetyl)amino]benzoate

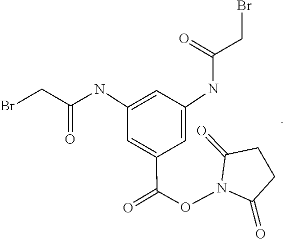

9. An intermediate compound of Formula III: 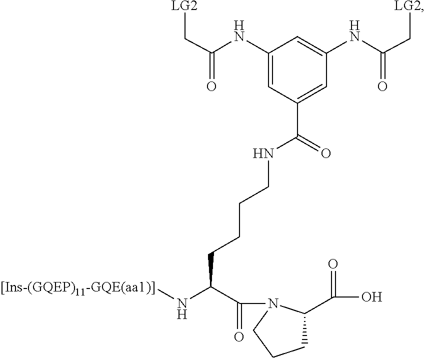
wherein Ins represents an analogue of human insulin, wherein said insulin analogue is a two-chain insulin molecule comprising an A- and a B-chain, and wherein said insulin analogue comprises the amino acid substitutions A14E, A21G, B25H, B29R, and the deletion desB30;
wherein (GQEP)₁₁-GQE-(aa1) is a recombinant extension fused to the C-terminus of the insulin A-chain;
wherein (aa1) is absent or proline (P); and
wherein each LG2 represents the leaving group of a thiol reactive group.

10. The intermediate compound of claim 9, wherein the insulin analogue further comprises the amino acid substitution B16H or B16E.

11. An intermediate compound which is 226A, 227G, 228P, 234A, 235K, 297E, 315Q, 384Q, des447 hIgG4-Fc(226-447).

12. A pharmaceutical composition comprising the insulin-Fc conjugate according to claim 1, and one or more pharmaceutically acceptable carriers or diluents.

13. An insulin-Fc conjugate according to claim 1 for use as a medicament.

14. A method of treatment or alleviation of a disease or disorder or condition in a human, which method comprises the step of administering to such a living animal a therapeutically effective amount of an insulin-Fc conjugate according to claim 1, wherein said disease, disorder or condition is selected from a disease, disorder or condition relating to diabetes, impaired glucose tolerance, hyperglycemia, dyslipidemia, obesity, metabolic syndrome, hypertension, cognitive disorders, atherosclerosis, myocardial infarction, stroke, cardiovascular disorders, coronary heart disease, inflammatory bowel syndrome, dyspepsia, or gastric ulcers.

15. (canceled)

16. The method according to claim 14, wherein the disease, disorder or condition is Type 1 diabetes or Type 2 diabetes.

17. The method according to claim 14, wherein the disease, disorder or condition is metabolic syndrome X or insulin resistance syndrome.


SEQUENCE LISTING (12 sequences)

SEQ ID NO: 1 - protein, 21 aa, ARTIFICIAL
  Human insulin A-chain
GIVEQCCTSICSLYQLENYCN

SEQ ID NO: 2 - protein, 30 aa, ARTIFICIAL
  Human insulin B-chain
FVNQHLCGSHLVEALYLVCGERGFFYTPKT

SEQ ID NO: 3 - protein, 21 aa, ARTIFICIAL
  A14E, A21G human insulin A-chain
GIVEQCCTSICSLEQLENYCG

SEQ ID NO: 4 - protein, 29 aa, ARTIFICIAL
  B25H, B29R, desB30 human insulin B-chain
FVNQHLCGSHLVEALYLVCGERGFHYTPR

SEQ ID NO: 5 - protein, 29 aa, ARTIFICIAL
  B16H, B25H, B29R, desB30 human insulin B-chain
FVNQHLCGSHLVEALHLVCGERGFHYTPR

SEQ ID NO: 6 - protein, 29 aa, ARTIFICIAL
  B16E, B25H, B29R, desB30 human insulin B-chain
FVNQHLCGSHLVEALELVCGERGFHYTPR

SEQ ID NO: 7 - protein, 221 aa, ARTIFICIAL
  226A, 227G, 228P, 234A, 235K, 297E, 315Q, 384Q, des447 hIgG4-Fc(226-447)
AGPCPAPEAKGGPSVFLFPPKPKDTLMISRTPEVTCVVVDVSQEDPEVQFNWYVDGVEVHNAKTKPREEQFESTYRVVSVLTVLHQDWLQGKEYKCKVSNKGLPSSIEKTISKAKGQPREPQVYTLPPSQEEMTKNQVSLTCLVKGFYPSDIAVEWESQGQPENNYKTTPPVLDSDGSFFLYSRLTVDKSRWQEGNVFSCSVMHEALHNHYTQKSLSLSLG

SEQ ID NO: 8 - protein, 6 aa, ARTIFICIAL
  hinge core sequence (positions 226-231) of native human IgG4
CPSCPA

SEQ ID NO: 9 - protein, 6 aa, ARTIFICIAL
  hinge core sequence
AGPCPA

SEQ ID NO: 10 - protein, 71 aa, ARTIFICIAL
  A14E, A21G, A22(GQEP)12, A70K, A71P human insulin
GIVEQCCTSICSLEQLENYCGGQEPGQEPGQEPGQEPGQEPGQEPGQEPGQEPGQEPGQEPGQEPGQEPKP

SEQ ID NO: 11 - protein, 70 aa, ARTIFICIAL
  A14E, A21G, A22(GQEP)11, A66G, A67Q, A68E, A69K, A70P human insulin
GIVEQCCTSICSLEQLENYCGGQEPGQEPGQEPGQEPGQEPGQEPGQEPGQEPGQEPGQEPGQEPGQEKP

SEQ ID NO: 12 - protein, 221 aa, ARTIFICIAL
  wt IgG4-Fc(227-447)
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK